(19) United States
(12) Patent Application Publication
(10) Pub. No.: US 2025/0003949 A1
(43) Pub. Date: Jan. 2, 2025

(54) CELL SAMPLE MEASUREMENT METHOD AND CELL SAMPLE MEASUREMENT APPARATUS

(71) Applicant: Hamamatsu Photonics K.K., Hamamatsu-shi (JP)

(72) Inventors: Tokio TAKAGI, Hamamatsu-shi (JP); Toyohiko YAMAUCHI, Hamamatsu-shi (JP); Tatsuo DOUGAKIUCHI, Hamamatsu-shi (JP); Yoichi KAWADA, Hamamatsu-shi (JP); Yoshiyuki SHIMIZU, Hamamatsu-shi (JP); Gen TAKEBE, Hamamatsu-shi (JP)

(73) Assignee: Hamamatsu Photonics K.K., Hamamatsu-shi (JP)

(21) Appl. No.: 18/739,556

(22) Filed: Jun. 11, 2024

(30) Foreign Application Priority Data
Jun. 27, 2023 (JP) 2023-104916

(51) Int. Cl. G01N 33/483; G01N 1/28; G01N 1/30; G01N 21/64

(52) U.S. Cl. CPC G01N 33/4833; G01N 1/2813; G01N 1/30; G01N 21/6428; G01N 2021/6439

(57) ABSTRACT

A cell sample measurement apparatus includes a sample stage for placing an object cell sample containing a plurality of cells cultured on a culture dish, an evaluation light irradiation unit for irradiating N irradiation regions set by dividing a culture region in the object cell sample with evaluation light having a wavelength in a mid-infrared region with irradiation amounts different from each other, a dead cell number measurement unit for measuring a dead cell number generated according to the irradiation amount of the evaluation light in each of the N irradiation regions, and a culture state analysis unit for obtaining, for the object cell sample, an object correlation between the irradiation amount of the evaluation light and the dead cell number for the N irradiation regions serving as an index of evaluation of a culture state of the object cell sample.

### CROSS-REFERENCE TO RELATED APPLICATION

This application is based upon and claims the benefit of priority from Japanese Patent Application No. 2023-104916, filed on Jun. 27, 2023, the entire contents of which are incorporated herein by reference.

### TECHNICAL FIELD

The present disclosure relates to a cell sample measurement method and a cell sample measurement apparatus for performing a measurement of a culture state of a cell sample containing a plurality of cells cultured on a culture dish.

### BACKGROUND

A technique for culturing cells is important not only in a basic research on cells, but also in a practical field such as, for example, regenerative medicine. In the culture of the cells, in order to further proliferate a cell system and a cell line, an operation called a passage is performed in which a culture medium is removed from the cell culture system, and the cultured cells are transferred to a new culture medium.

Further, the number of times of performing the passage operation in the cell sample in which a plurality of cells are cultured is referred to as a passage number.
Patent Document 1: Japanese Patent Application Laid-Open Publication No. 2019-150018
Patent Document 2: Japanese Patent Application Laid-Open Publication No. 2018-130093
Non Patent Document 1: P. Hughes et al., “The costs of using unauthenticated, over-passaged cell lines: how much more data do we need?”, Bio Techniques Vol. 43 (2007), pp. 575-586
Non Patent Document 2: E. Larson et al., “Mid-infrared absorption by soft tissue sarcoma and cell ablation utilizing a mid-infrared interband cascade laser”, J. Biomed. Opt. Vol. 26 (2021), pp. 043012-1-043012-10
Non Patent Document 3: N. Malpica et al., “Applying Watershed Algorithms to the Segmentation of Clustered Nuclei”, Cytometry Vol. 28 (1997), pp. 289-297

### SUMMARY

In the cell sample obtained by culturing the plurality of cells as described above, the passage number, a condition of each passage operation, and the like affect a quality, a culture state, and the like of the cells grown in the cell sample (see, for example, Patent Document 1 and Non Patent Document 1). That is, in the passage operation of the cell sample, in many cases, the cultured cells are subjected to damage, stress, and the like by being transferred to a new environment, and thus, the quality and the state of the cells deteriorate.

In general, it is known that, as the passage number increases, the quality of the cultured cells deteriorates due to a change in differentiation and proliferation capacity, a change in form, a change in gene, and the like. Therefore, the culture state such as the passage number, the passage condition and the like of the cell sample is very important information in performing an experiment, a measurement, and the like using the cell sample. However, a technique for measuring and evaluating the culture state of the cells in the cell sample described above has not been established until now.

An object of the present invention is to provide a cell sample measurement method and a cell sample measurement apparatus capable of suitably performing a measurement of a culture state of a cell sample containing a plurality of cells.

The present inventors have studied effects of activation of the cells and the like by light irradiation performed using laser light supplied from a quantum cascade laser (QCL) and the like on the cell sample in which the plurality of cells are cultured. In the above research on the effects of the light irradiation on the cell sample, the present inventors have found that dead cells are generated when the light irradiation is performed at an irradiation amount of a certain level or more. In addition, when cell death due to the light irradiation described above was examined in detail, it was found that the generation of dead cells depends on the culture state such as the passage number of the cell sample, and the present invention has been achieved.

An embodiment of the present invention is a cell sample measurement method. The cell sample measurement method includes (1) a preparation step of placing an object cell sample of a measurement object containing a plurality of cells cultured on a culture dish on a sample stage; (2) an evaluation light irradiation step of irradiating N irradiation regions (N is an integer of 2 or more) set by dividing a culture region of the plurality of cells in the object cell sample with evaluation light having a wavelength in a mid-infrared region supplied from an irradiation light source with irradiation amounts different from each other; (3) a dead cell number measurement step of measuring a dead cell number generated according to the irradiation amount of the evaluation light in each of the N irradiation regions of the object cell sample; and (4) a culture state analysis step of obtaining, for the object cell sample, an object correlation between the irradiation amount of the evaluation light and the dead cell number for the N irradiation regions serving as an index of evaluation of a culture state of the object cell sample.

An embodiment of the present invention is a cell sample measurement apparatus. The cell sample measurement apparatus includes (1) a sample stage for placing an object cell sample of a measurement object containing a plurality of cells cultured on a culture dish; (2) an evaluation light irradiation unit for irradiating N irradiation regions (N is an integer of 2 or more) set by dividing a culture region of the plurality of cells in the object cell sample with evaluation light having a wavelength in a mid-infrared region supplied from an irradiation light source with irradiation amounts different from each other; (3) a dead cell number measurement unit for measuring a dead cell number generated according to the irradiation amount of the evaluation light in each of the N irradiation regions of the object cell sample; and (4) a culture state analysis unit for obtaining, for the object cell sample, an object correlation between the irradiation amount of the evaluation light and the dead cell number for the N irradiation regions serving as an index of evaluation of a culture state of the object cell sample.

In the cell sample measurement method and the cell sample measurement apparatus described above, the evaluation light having the wavelength in the mid-infrared region is used in the measurement of the object cell sample containing the plurality of cells cultured on the single culture dish, and the N irradiation regions set by dividing the culture region in the object cell sample are irradiated with the evaluation light with the irradiation amounts different from each other, and further, for each of the N irradiation regions, the dead cell number (the number of dead cells) generated according to the irradiation amount of the evaluation light is measured. In addition, based on the above measurement result, the object correlation between the irradiation amount of the evaluation light and the dead cell number for the N irradiation regions is obtained for the object cell sample.

According to the above configuration, for the object cell sample in which the plurality of cells are cultured on the culture dish, the plurality of irradiation regions are set by dividing the culture region, and the irradiation regions are respectively irradiated with the evaluation light with the irradiation amounts different from each other, and thus, it is possible to suitably measure the object correlation between the irradiation amount of the evaluation light and the dead cell number, which can be used as the index of the evaluation of the culture state of the cells in the object cell sample.

According to the cell sample measurement method and the cell sample measurement apparatus of the present invention, it is possible to suitably perform the measurement for the culture state of the cell sample containing the plurality of cells.

The present invention will be more fully understood from the detailed description given hereinbelow and the accompanying drawings, which are given by way of illustration only and are not to be considered as limiting the present invention.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will be apparent to those skilled in the art from this detailed description.

### BRIEF DESCRIPTION OF THE DRAWINGS

FIG. 1 is a diagram schematically illustrating a configuration of a cell sample measurement apparatus according to an embodiment.

FIG. 2 is a block diagram illustrating an example of a configuration of a culture state analysis unit in the cell sample measurement apparatus illustrated in FIG. 1.

FIG. 3A and FIG. 3B are diagrams illustrating an example of setting of an irradiation amount of evaluation light supplied from an irradiation light source.

FIG. 4 is a graph showing an example of setting of an irradiation power of the evaluation light supplied from the irradiation light source.

FIG. 5 is a diagram illustrating an example of a plurality of irradiation regions set by dividing a culture region in an object cell sample.

FIG. 6 is a diagram illustrating irradiation of the evaluation light for irradiation points set in the plurality of irradiation regions illustrated in FIG. 5.

FIG. 7 is a diagram illustrating a configuration of an evaluation light irradiation unit in the cell sample measurement apparatus illustrated in FIG. 1 according to a first modification.

FIG. 8 is a diagram illustrating a configuration of the evaluation light irradiation unit in the cell sample measurement apparatus illustrated in FIG. 1 according to a second modification.

FIG. 9 is a diagram schematically illustrating a fluorescent image of the object cell sample stained with a fluorescent dye for selectively staining dead cells.

FIG. 10 is a diagram illustrating an example of a configuration of a dead cell number measurement unit in the cell sample measurement apparatus illustrated in FIG. 1.

FIG. 11 is a diagram showing an example of the fluorescent image generated by irradiating the cell sample stained with the fluorescent dye with excitation light.

FIG. 12A to FIG. 12C are graphs each showing a correlation between an irradiation time of the evaluation light and a dead cell number for each of the cell samples with passage numbers 45, 53, and 55.

FIG. 13 is a graph showing the correlation between the irradiation time of the evaluation light and the dead cell number for each of the cell samples with the passage numbers 45, 53, and 55.

FIG. 14A and FIG. 14B are graphs showing a relationship between a slope of an approximate straight line of the correlation between the irradiation time of the evaluation light and the dead cell number, and the passage number of the cell sample.

FIG. 15A and FIG. 15B are graphs showing a relationship between a dead cell number at a predetermined irradiation time in the correlation between the irradiation time of the evaluation light and the dead cell number, and the passage number of the cell sample.

FIG. 16A and FIG. 16B are graphs each showing the correlation between the irradiation time and the irradiation power of the evaluation light and the dead cell number.

FIG. 17 is a graph showing the correlation between the irradiation time and the irradiation power of the evaluation light and the dead cell number.

FIG. 18 is a graph showing the correlation between the irradiation time and the irradiation power of the evaluation light and the dead cell number.

### DETAILED DESCRIPTION

Hereinafter, embodiments of a cell sample measurement method and a cell sample measurement apparatus will be described in detail with reference to the accompanying drawings. In the description of the drawings, the same elements will be denoted by the same reference signs, and redundant description will be omitted. Further, the dimensional ratios in the drawings are not always coincident with those in the description.

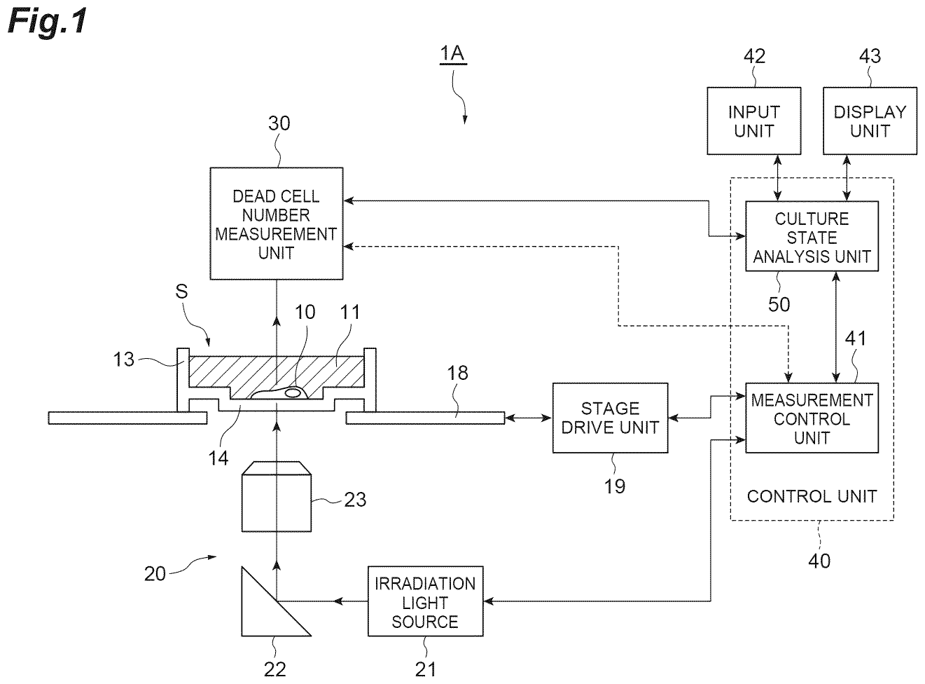

FIG. 1 is a diagram schematically illustrating a configuration of a cell sample measurement apparatus according to an embodiment. The cell sample measurement apparatus 1A according to the present embodiment is a measurement apparatus for performing a measurement of a culture state of cells in an object cell sample S containing a plurality of cells of the same type cultured in a culture region on a single culture dish 13 which is a culture container, and includes a sample stage 18, an evaluation light irradiation unit 20, a dead cell number measurement unit 30, and a control unit 40. The object cell sample S serving as a measurement object by the measurement apparatus 1A is, for example, a cell sample with an unknown culture state such as a passage number or the like. In addition, a reference cell sample with a known culture state may be set as the object cell sample S of the measurement object.

In the object cell sample S illustrated in FIG. 1, in the culture region on the culture dish 13, a plurality of cells 10 are cultured in a state of being contained in the culture dish 13 together with a culture solution 11. Further, in FIG. 1, with respect to the plurality of cells 10 contained in the object cell sample S, a single cell 10 in the culture solution 11 is illustrated for the sake of simplicity of illustration.

The object cell sample S is placed on the sample stage 18. In the present embodiment, when an irradiation optical axis of evaluation light by the evaluation light irradiation unit 20 is set to a z axis, the sample stage 18 is arranged in parallel to an xy plane perpendicular to the z axis. Further, the sample stage 18 is configured to be movable in the xy plane perpendicular to the irradiation optical axis of the evaluation light. In addition, in general, the sample stage 18 may be arranged in parallel to a plane intersecting with the irradiation optical axis of the evaluation light, and configured to be movable in the above plane.

Further, a stage drive unit 19 is provided for the sample stage 18 described above. The stage drive unit 19 drives the sample stage 18 on which the object cell sample S is placed in the xy plane (in the plane intersecting with the irradiation optical axis), thereby setting and controlling an irradiation position of the evaluation light in the culture region of the object cell sample S. In addition, as a drive mechanism of the sample stage 18, for example, a stepping motor, piezo actuator, or the like can be used.

The evaluation light irradiation unit (evaluation light irradiation apparatus) 20 is arranged below the sample stage 18 on which the object cell sample S is placed, and is optically coupled to a bottom surface 14 of the culture dish 13 in the object cell sample S via an opening provided at a predetermined position of the sample stage 18. In the present embodiment, the evaluation light irradiation unit 20 includes an irradiation light source 21, a reflection mirror 22, and an objective lens 23.

The evaluation light irradiation unit 20 irradiates an irradiation region set in the culture region of the plurality of cells 10 in the object cell sample S with the evaluation light having a predetermined wavelength in a mid-infrared region under a predetermined irradiation condition. The evaluation light supplied from the irradiation light source 21 is reflected by the reflection mirror 22, and applied to the plurality of cells 10 contained in the object cell sample S via the objective lens 23 and the bottom surface 14 of the culture dish 13 along the predetermined irradiation optical axis.

Specifically, in the object cell sample S, the evaluation light irradiation unit 20 sets N irradiation regions (a plurality of irradiation regions, N is an integer of 2 or more) by dividing the culture region of the plurality of cells 10 on the culture dish 13. Further, the N irradiation regions described above are irradiated with the evaluation light supplied from the irradiation light source 21 with irradiation amounts different from each other.

The dead cell number measurement unit (dead cell number measurement apparatus) 30 is arranged above the sample stage 18 on which the object cell sample S is placed. The dead cell number measurement unit 30 measures the number of dead cells (the dead cell number) generated according to the irradiation amount of the evaluation light by the evaluation light irradiation unit 20 in the irradiation region of the object cell sample S. In the case in which the N irradiation regions are set in the culture region of the object cell sample S as described above, the dead cell number measurement unit 30 measures the dead cell number generated according to the irradiation amount of the evaluation light in each of the N irradiation regions of the object cell sample S. The specific configuration of the dead cell number measurement unit 30 is appropriately set according to a measurement method of the number of dead cells in the dead cell number measurement unit 30.

In addition, in the configuration of FIG. 1, the configuration in which the evaluation light irradiation unit 20 and the dead cell number measurement unit 30 are integrally provided in the measurement apparatus 1A including the sample stage 18 is illustrated, and further, the configuration is not limited thereto. For example, the dead cell number measurement unit 30 may be configured to be provided separately from the evaluation light irradiation unit 20. In this case, a sample stage in the dead cell number measurement unit 30 is provided separately from the sample stage 18 in the evaluation light irradiation unit 20. Further, the details of the irradiation condition of the evaluation light by the evaluation light irradiation unit 20, the configuration of the dead cell number measurement unit 30, the measurement method of the dead cell number, and the like will be described later.

The control unit 40 controls the operations of respective parts of the sample stage 18, the evaluation light irradiation unit 20, and the dead cell number measurement unit 30 in the measurement apparatus 1A, and further, performs necessary analysis on the measurement result by the evaluation light irradiation unit 20 and the dead cell number measurement unit 30. In the present embodiment, the control unit 40 includes a measurement control unit 41 and a culture state analysis unit 50.

The measurement control unit (measurement control apparatus) 41 controls the drive operation of the sample stage 18 by the stage drive unit 19, and the irradiation operation of the evaluation light on the object cell sample S by the evaluation light irradiation unit 20 (for example, the supply operation of the evaluation light by the irradiation light source 21). Further, in the case in which the dead cell number measurement unit 30 is provided integrally with the evaluation light irradiation unit 20, the measurement control unit 41 may be configured to further control the measurement operation of the dead cell number in the object cell sample S by the dead cell number measurement unit 30, as illustrated by a dashed arrow in FIG. 1.

The culture state analysis unit 50 is a culture state analysis apparatus for analyzing the culture state of the object cell sample S containing the plurality of cells 10. Further, in the configuration illustrated in FIG. 1, an input unit 42 and a display unit 43 are provided for the culture state analysis unit 50. The input unit 42 is used for inputting information necessary for the analysis of the culture state of the object cell sample S and the like. The display unit 43 is used for displaying an analysis condition and an analysis result of the culture state of the object cell sample S, the measurement result by the evaluation light irradiation unit 20 and the dead cell number measurement unit 30, and the like.

In the control unit 40, each of the measurement control unit 41 and the culture state analysis unit 50 may be constituted by, for example, a computer including a CPU as a processing unit and a ROM, a RAM, an external storage device, and the like as a storage unit. The measurement control unit 41 and the culture state analysis unit 50 may be constituted by separate computers, or may be constituted by a single computer. Further, in the case in which the measurement control unit 41 is provided separately from the culture state analysis unit 50, the measurement control unit 41 may be provided with an input unit and a display unit as in the case of the culture state analysis unit 50.

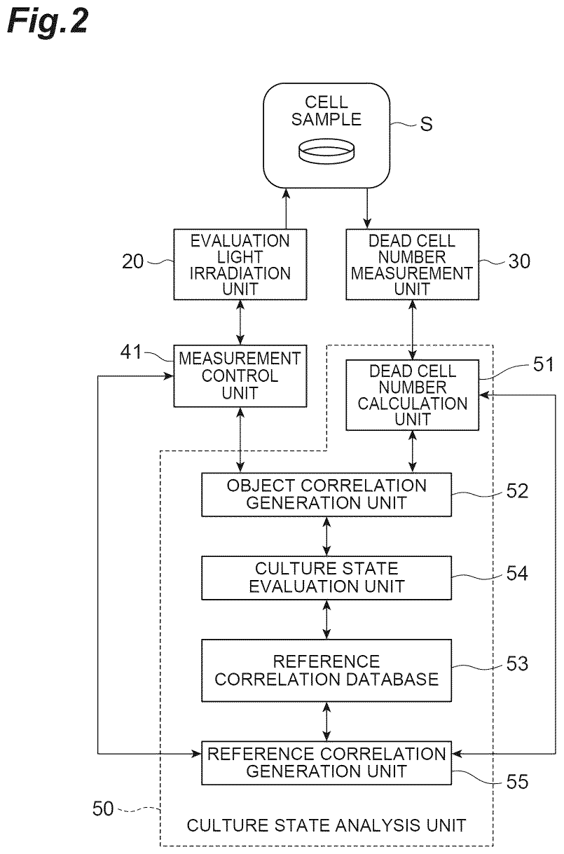

FIG. 2 is a block diagram illustrating an example of a configuration of the culture state analysis unit 50 in the cell sample measurement apparatus 1A illustrated in FIG. 1. The culture state analysis unit (culture state analysis apparatus) 50 according to the present configuration example includes a dead cell number calculation unit 51, an object correlation generation unit 52, a reference correlation database 53, a culture state evaluation unit 54, and a reference correlation generation unit 55.

In addition, the present configuration example illustrates the configuration of the culture state analysis unit 50 in the case in which the cell sample measurement apparatus 1A is configured as a cell sample evaluation apparatus for performing the measurement of the culture state of the cells in the object cell sample S, and in addition, performing the evaluation of the culture state of the object cell sample S. In the case in which only the measurement is performed without performing the evaluation of the culture state of the object cell sample S, it is not necessary to provide the reference correlation database 53, the culture state evaluation unit 54, and the reference correlation generation unit 55.

The dead cell number calculation unit 51 calculates the dead cell number generated in the irradiation region of the object cell sample S based on the measurement data of the dead cell number input from the dead cell number measurement unit 30. In the case in which the N irradiation regions are set for the object cell sample S, the dead cell number calculation unit 51 calculates the dead cell number generated in each of the N irradiation regions. In addition, in the case in which the dead cell number is directly acquired in the measurement data by the dead cell number measurement unit 30, it is not necessary to provide the dead cell number calculation unit 51.

The object correlation generation unit 52 obtains an object correlation between the irradiation amount of the evaluation light and the dead cell number for the object cell sample S based on the measurement result including the irradiation data of the evaluation light for the irradiation region input from the evaluation light irradiation unit 20 or the measurement control unit 41 and the measurement data of the dead cell number input from the dead cell number measurement unit 30 or the dead cell number calculation unit 51. In the case in which the N irradiation regions are set for the object cell sample S, the object correlation generation unit 52 obtains the object correlation between the irradiation amount of the evaluation light and the dead cell number for the N irradiation regions. The object correlation described above serves as an index of the evaluation of the culture state of the cells in the object cell sample S.

The reference correlation database 53 is a reference correlation storage unit for storing a reference correlation between the irradiation amount of the evaluation light and the dead cell number for a reference cell sample with a known culture state of the cells. In the case in which a single reference cell sample is assumed, the reference correlation database 53 stores a single reference correlation. Further, in the case in which a plurality of reference cell samples with culture states different from each other are assumed, the reference correlation database 53 stores a plurality of reference correlations for the reference cell samples.

The culture state evaluation unit 54 compares the reference correlation stored in the reference correlation database 53 and the object correlation obtained by the object correlation generation unit 52, and evaluates the culture state of the cells in the object cell sample S based on the comparison result. In the case in which the plurality of reference correlations are stored in the reference correlation database 53, the culture state evaluation unit 54 evaluates the culture state of the cells in the object cell sample S by comparing each of the plurality of reference correlations and the object correlation.

In the configuration example illustrated in FIG. 2, in the culture state analysis unit 50, the reference correlation generation unit 55 is provided in addition to the reference correlation database 53. The reference correlation generation unit 55 obtains, for the reference cell sample, in the same manner as the object cell sample, the reference correlation between the irradiation amount of the evaluation light and the dead cell number based on the measurement result obtained by the evaluation light irradiation unit 20 and the dead cell number measurement unit 30. In addition, in the case in which the reference correlation is acquired in advance and stored in the reference correlation database 53, it is not necessary to provide the reference correlation generation unit 55.

A cell sample measurement method performed by the cell sample measurement apparatus 1A illustrated in FIG. 1 and FIG. 2 includes a preparation step, an evaluation light irradiation step, a dead cell number measurement step, and a culture state analysis step. In the preparation step, the object cell sample S containing the plurality of cells 10 cultured on the single culture dish 13 is placed on the sample stage 18. In the evaluation light irradiation step, the evaluation light irradiation unit 20 irradiates the N irradiation region set by dividing the culture region in the object cell sample S with the evaluation light supplied from the irradiation light source 21 with irradiation amounts different from each other.

In the dead cell number measurement step, the dead cell number measurement unit 30 measures the dead cell number generated according to the irradiation amount of the evaluation light in each of the N irradiation regions of the object cell sample S irradiated with the evaluation light by the evaluation light irradiation unit 20. In the culture state analysis step, the culture state analysis unit 50 obtains the object correlation between the irradiation amount of the evaluation light and the dead cell number for the N irradiation regions serving as the index of the evaluation of the culture state of the object cell sample S. Further, in the case in which the evaluation of the culture state of the object cell sample S is performed, in the culture state analysis step, the culture state of the object cell sample S is evaluated by comparing the reference correlation for the reference cell sample and the object correlation described above.

The effects of the cell sample measurement apparatus 1A and the cell sample measurement method according to the above embodiment will be described.

In the measurement apparatus 1A and the measurement method illustrated in FIG. 1 and FIG. 2, the evaluation light having the wavelength in the mid-infrared region is used in the measurement of the object cell sample S containing the plurality of cells 10 cultured on the culture dish 13, and the N irradiation regions set by dividing the culture region in the object cell sample S are irradiated with the evaluation light with the irradiation amounts different from each other by the evaluation light irradiation unit 20, and further, the dead cell number (the number of dead cells) generated in each of the irradiation regions according to the irradiation amount of the evaluation light is measured by the dead cell number measurement unit 30. In addition, based on the above measurement result, in the culture state analysis unit 50, the object correlation between the irradiation amount of the evaluation light and the dead cell number for the N irradiation regions is obtained for the object cell sample S.

According to the configuration described above, for the object cell sample S in which the plurality of cells 10 are cultured on the culture dish 13, the plurality of irradiation regions are set in the culture region, and the irradiation regions are respectively irradiated with the evaluation light with the irradiation amounts different from each other, and thus, it is possible to suitably measure the object correlation between the irradiation amount of the evaluation light and the dead cell number, which can be used as the index of the evaluation of the culture state of the cells in the object cell sample S. Further, it is preferable that the number N of the irradiation regions obtained by dividing the culture region is set appropriately according to the specific content of the object correlation to be acquired.

In addition, in Non Patent Document 2 (E. Larson et al., J. Biomed. Opt. Vol. 26 (2021), pp. 043012-1-043012-10), it is described that the cell sample is irradiated with the laser light in the mid-infrared region to examine a generation threshold value of dead cells. However, Non Patent Document 2 does not describe or suggest that there is a correlation between the generation of dead cells in the cell sample and the culture state such as the passage number of the cell sample.

In the measurement apparatus 1A and the measurement method of the above embodiment, in the evaluation light irradiation unit 20, a plurality of irradiation points may be set for irradiating with the evaluation light in each of the N irradiation regions, and the dead cell number measurement unit 30 may measure the dead cell number at each of the plurality of irradiation points in each of the N irradiation regions.

As described above, the plurality of irradiation points are set in each of the N irradiation regions set in the culture region, and the irradiation of the evaluation light and the measurement of the dead cell number are performed at each of the irradiation points, and thus, the object correlation between the irradiation amount of the evaluation light and the dead cell number can be suitably and accurately measured.

Further, in the case in which the plurality of irradiation points are set in each of the irradiation regions as described above, in the evaluation light irradiation unit 20, all the irradiation points set in the N irradiation regions may be arranged in a grid pattern in the culture region, and the irradiation points may be sequentially irradiated with the evaluation light. Further, the culture state analysis unit 50 may obtain the object correlation between the irradiation amount of the evaluation light in each irradiation region and an average value of the dead cell numbers at the plurality of irradiation points in each irradiation region for the N irradiation regions. The details of the setting and the like of the irradiation region and the irradiation point described above will be described later.

Further, in the measurement apparatus 1A and the measurement method illustrated in FIG. 1 and FIG. 2, the evaluation light having the wavelength in the mid-infrared region is used, and the irradiation region set in the object cell sample S is irradiated with the evaluation light by the evaluation light irradiation unit 20, and further, the dead cell number (the number of dead cells) generated in the irradiation region according to the irradiation amount of the evaluation light is measured by the dead cell number measurement unit 30. In addition, based on the above measurement result, in the culture state analysis unit 50, the object correlation between the irradiation amount of the evaluation light and the dead cell number is obtained for the object cell sample S, and further, the reference correlation prepared for the reference cell sample may be compared with the object correlation to evaluate the culture state of the cells contained in the object cell sample S. In this case, the measurement apparatus 1A functions as the evaluation apparatus as described above.

According to the configuration described above, for the object cell sample S in which the plurality of cells 10 are cultured on the culture dish 13, it is possible to suitably evaluate the culture state of the cells in the object cell sample S using the object correlation between the irradiation amount of the evaluation light and the dead cell number as the index. In addition, it is preferable that the irradiation condition of the evaluation light for the irradiation region of the object cell sample S is appropriately set such that the object correlation between the irradiation amount of the evaluation light and the dead cell number used for the evaluation of the object cell sample S can be acquired.

In the measurement apparatus 1A and the measurement method of the above embodiment, the culture state evaluation unit 54 of the culture state analysis unit 50 preferably evaluates, specifically, the culture state including at least one of the passage number, the passage condition (the condition of the passage operation), and the culture condition between the passages of the object cell sample S as the culture state of the cells in the object cell sample S. In this case, the object correlation obtained in the object correlation generation unit 52 is used as the index of the evaluation of the culture state including at least one of the passage number, the passage condition, and the culture condition between the passages of the object cell sample S

As described above, for the object cell sample S, the object correlation between the irradiation amount of the evaluation light and the dead cell number described above is used as the index of the evaluation, and thus, the culture state such as the passage number and the like of the object cell sample S can be suitably evaluated. Further, in the case in which the passage condition and the culture condition between the passages are considered to be relatively constant, the case in which the information of the passage number is considered to be important, or the like, in particular, the passage number may be evaluated as the culture state of the cells in the object cell sample S. In this case, the object correlation is used as the index of the evaluation of the passage number of the object cell sample S.

In the measurement apparatus 1A and the measurement method of the above embodiment, as described above, the reference correlation database 53 of the culture state analysis unit 50 may store the plurality of reference correlations for the plurality of reference cell samples with the culture states different from each other, and the culture state evaluation unit 54 may evaluate the culture state of the object cell sample S by comparing each of the plurality of reference correlations and the object correlation.

As described above, by preparing the plurality of reference correlations for the plurality of reference cell samples with the known culture states and the culture states different from each other in the reference correlation database 53, and comparing each of the reference correlations and the object correlation, it is possible to accurately evaluate the culture state of the object cell sample S in more detail.

In the measurement apparatus 1A and the measurement method of the above embodiment, the culture state evaluation unit 54 of the culture state analysis unit 50 may compare, in the comparison between the reference correlation and the object correlation, at least one of a parameter representing an approximate straight line or an approximate curve of the correlation, a dead cell number at a predetermined irradiation amount, and a threshold irradiation amount for generating dead cells.

As described above, for the correlation between the irradiation amount of the evaluation light and the dead cell number, specifically, by comparing the reference correlation and the object correlation by using at least one of the parameter representing the approximate straight line or the approximate curve of the correlation, the dead cell number at the predetermined irradiation amount, and the threshold irradiation amount for the dead cell generation, it is possible to suitably evaluate the culture state of the object cell sample S.

In the measurement apparatus 1A and the measurement method of the above embodiment, as described above, the culture state analysis unit 50 may include the reference correlation generation unit 55 for obtaining, for the reference cell sample, the reference correlation between the irradiation amount of the evaluation light and the dead cell number based on the measurement result obtained by using the evaluation light irradiation unit 20 and the dead cell number measurement unit 30.

As described above, for the reference cell sample, by using the configuration in which the reference correlation between the irradiation amount of the evaluation light and the dead cell number is acquired using the same apparatus and method as those for the object cell sample S of the measurement object, it is possible to suitably realize the evaluation of the culture state of the object cell sample S by the comparison between the reference correlation and the object correlation. In addition, for the reference cell sample, the reference correlation may be acquired using an apparatus different from that for the object cell sample S. In this case, it is necessary to match the measurement conditions such as the irradiation conditions of the evaluation light between the reference cell sample and the object cell sample. Further, for the reference correlation, it may be acquired in advance and stored in the reference correlation database 53.

The configuration of the measurement apparatus 1A and the measurement method according to the above embodiment will be further described. First, the object cell sample S, which is the measurement object for the culture state, the type of the cells contained in the cell sample, and the like will be described.

The cells used in the research, experiment, and the like related to the cells are roughly classified into established cell lines and primary cultured cells. The established cell line is a cell which has become able to proliferate indefinitely without being restricted by a cell cycle. Further, the primary cultured cell is a cell directly collected from a tissue and cultured. In general, in the primary cultured cell, when a cell division is repeated, the cell division stops at a finite number of times due to the Hayflick limit. However, in the primary culture, a chromosomal abnormality is rarely caused, and an infinitely proliferating cell may appear in some cases. This is a so-called immortalization of the cells, and such cells are called the established cell lines.

Further, the cells used in the research and the like are roughly classified into adherent cells and floating cells from the viewpoint of the culture method. The adherent cells grow while adhering to the culture container, and on the other hand, the floating cells grow in a culture medium in a floating state. The cell sample measurement by the measurement apparatus 1A described above is considered to be difficult to apply to the cell sample containing the floating cells in consideration of the optical system, and it is preferable to use the cell sample containing the adherent cells as the object cell sample S of the measurement object.

In general, mammalian cell lines such as CHO, HeLa, MCF-7, 293, 3T3, COS, and the like are known as examples of the established adherent cell lines, and are widely used in molecular biological research, biochemical research, and the like. Further, in an example of the cell sample measurement described later, the C6 cells which are the established adherent cell line derived from rat glioma are used. In addition, the well-known cell line as described above has been established for a long time, and thus, the passage number of the cell line is often unknown.

Further, stem cells such as embryonic stem cells (ES cells), induced pluripotent stem cells (iPS cells), embryonic carcinoma cells (EC cells), mesenchymal stem cells, and the like are more difficult to culture than general cell lines, and specific techniques are required to perform stable culture. In the use of the above stem cells, it is important to accurately evaluate the culture state such as the passage number.

On the other hand, the primary cultured cells accurately reproduce a physiological state of the cells in a body, and thus, it is possible to obtain appropriate data indicating a state of a living body, and the primary cultured cells are used in preclinical research, biological research, and the like. However, the primary cultured cell itself is very fragile, and thus, sufficient care should be taken when applying the cell sample measurement by the measurement apparatus 1A described above.

Further, in the object cell sample S, it is preferable that the plurality of cells 10 cultured on the culture dish 13 are uniformly distributed in the culture region. In the actual case, it is preferable to perform necessary measurement and evaluation for the object cell sample S by confirming that the cultured cells are in the state of being 100% confluent on the culture dish 13, that is, in the state in which the adherent cells cover an adhesion surface of the culture dish 13.

The culture dish 13 used for the culture of the cells in the object cell sample S will be described. In the configuration illustrated in FIG. 1, the evaluation light irradiation unit 20 performs the irradiation on the object cell sample S with the evaluation light from the bottom surface 14 side of the culture dish 13. In the above configuration, in order to prevent the mid-infrared light used as the evaluation light from being attenuated before reaching the cells 10, it is preferable to use a culture dish having the bottom surface 14 formed of a material with a high transmittance for the mid-infrared light as the culture dish 13.

Specifically, for example, for each of various commercially available culture dishes, an infrared absorption spectrum of the bottom surface is measured, and a culture dish having a high transmittance for the evaluation light may be selected and used. In the example described later, a film bottom dish (product name: lumox dish) manufactured by Sarstedt is used as the culture dish 13. In the above culture dish, a polymer film is provided on the bottom surface.

As the material of the bottom surface of the culture dish which is the culture container, for example, silicon, germanium, calcium fluoride, barium fluoride, zinc selenide, zinc sulfide, chalcogenide glass, and the like, which are known in general to have a high infrared transmittance, can be used in addition to the polymer film described above. Further, for the culture dish, it is preferable to select a culture dish having low water solubility and low cytotoxicity.

The evaluation light used for the irradiation on the object cell sample S in the evaluation light irradiation unit 20, and the irradiation light source 21 used for supplying the evaluation light will be described. In the above measurement apparatus 1A, light having a predetermined wavelength in the mid-infrared region is used as the evaluation light as described above. The energy of the mid-infrared light matches the energy of fundamental vibrations of many molecules. Therefore, the mid-infrared light has a large absorption coefficient for the molecules. The absorption coefficient of the mid-infrared light for the molecules described above is, for example, several orders of magnitude higher than that of near-infrared light or visible light.

The light energy required to induce a change of the cell state by light absorption depends on the absorption coefficient of the cells of the object. Therefore, by using the mid-infrared light as the evaluation light for the object cell sample S, it is possible to induce the change of the cell state with small irradiation energy compared with the case in which the near-infrared light or the visible light is used.

For example, when the near-infrared light or the visible light is used as the evaluation light, large light energy is required to induce the change of the cell state, and thus, heat generation caused by the light irradiation may damage cells or a culture environment around the irradiation point of the evaluation light. Further, in the case of the near-infrared light or the visible light, a large size high intensity laser light source or the like may be necessary as the irradiation light source 21, and thus, there is a problem that the size of the apparatus becomes large.

On the other hand, in the configuration in which the mid-infrared light is used as the evaluation light, it is possible to realize light absorption by directly providing the energy to the cell molecules of the object, or the fundamental vibration of water contained in the cells, and thus, it is possible to selectively induce the change of the cell state only at the object position with small light energy. In this case, for example, a longitudinal single mode laser light source having a wavelength corresponding to a specific absorption line, that is, a laser light source having a narrow oscillation spectrum band can be suitably used. Further, the evaluation light using the mid-infrared light requires small light energy, and thus, for example, the change of the cell state can be induced even in the case of using a compact semiconductor laser. In this case, it is possible to reduce the size of the apparatus.

Further, as to the wavelength of the mid-infrared light used as the evaluation light, specifically, for example, in the evaluation light irradiation unit 20, the wavelength of the evaluation light is preferably set in the wavelength region of 3 μm or more and 20 μm or less. In this case, the object correlation between the irradiation amount of the evaluation light and the dead cell number serving as the index of the evaluation of the culture state of the object cell sample S can be suitably acquired.

In the above wavelength region, for example, there is a lot of absorption of water in a 3 μm band, and effective heating through water is possible. Further, in a wavelength range from 10 μm to 20 μm, there are absorption lines specific to relatively large molecules, for example, such as a molecule containing a benzene ring. In addition, as to a wavelength region of a long wavelength exceeding 20 μm, it is considered that it is difficult to incorporate an appropriate light source into the measurement apparatus 1A described above.

As the irradiation light source 21 used for supplying the evaluation light in the evaluation light irradiation unit 20, for example, a quantum cascade laser (QCL) can be used. The quantum cascade laser is a monopolar type laser element for generating light by transitions of electrons between subbands by using a level structure including the subbands formed in a semiconductor quantum well structure, and can realize a high efficiency and high output operation by cascade-coupling emission layers of the quantum well structure in multiple stages via injection layers.

In the case in which the QCL is used in the irradiation light source 21, the irradiation light source 21 may be constituted by, for example, the QCL element, a cooling mechanism, and a collimating lens. As a specific example of the QCL element, a DFB-QCL (model number: L12004-2190H-C) having a wavelength of 4.6 μm manufactured by Hamamatsu Photonics K.K. can be used. The irradiation light source described above is preferably a transverse single mode light source, that is, a light source having high spatial coherence in order to realize a small focusing diameter. Further, in the irradiation light source, it is preferable to perform temperature control, for example, by a TEC in order to stabilize the wavelength and output of the evaluation light. Further, a secondary cooling mechanism using a fan or water cooling may be provided.

As the collimating lens, it is necessary to use a lens made of a material which transmits the evaluation light having the wavelength from 3 μm to 20 μm described above. As the material of the lens described above, for example, ZnSe, Ge, CaF₂, and the like can be used. Further, it is preferable to provide anti-reflection coating on both surfaces of the collimating lens such that the transmittance for the wavelength of the evaluation light is, for example, 95% or more. Further, a plurality of lenses may be used in combination as the collimating lens, and further, it is preferable to use a single aspheric lens capable of correcting the spherical aberration because loss of the evaluation light increases in the case of using the plurality of lenses.

Further, as the light source element in the irradiation light source 21, in addition to the QCL described above, for example, an interband cascade laser (ICL), a CO₂ laser, a CO laser, a free electron laser, and the like can be used. In addition, a wavelength conversion technique using an optical parametric oscillator (OPO) may be used. Further, in the case in which the QCL or the ICL is used, from the viewpoint that the longitudinal single mode oscillation is possible, the configuration is not limited to the DFB type configuration described above, and further, an external cavity type configuration may be used. In addition, as to the wavelength region of the evaluation light, for example, the QCL can supply light having a wavelength of about 4 μm to 20 μm, and the ICL can supply light having a wavelength of about 3 μm to 7 μm.

Further, as the evaluation light supplied from the irradiation light source 21, continuous light may be use, or pulsed light may be used. Further, as the evaluation light, a burst pulse including a plurality of light pulses and having a range which is defined by an envelope of a predetermined shape may be used.

The configuration of the irradiation optical system used for the irradiation of the evaluation light on the object cell sample S in the evaluation light irradiation unit 20 will be described. In the measurement apparatus 1A described above, the evaluation light from the irradiation light source 21 is reflected by 90° by the reflection mirror 22, and the object cell sample S is irradiated via the objective lens 23. In the above configuration, a height of an irradiation plane (operation plane) can be kept low by bending an optical path of the evaluation light. As the reflection mirror 22, for example, a gold coated flat mirror, a high reflection flat mirror by using a dielectric multilayer film, and the like can be used.

The configuration of the irradiation optical system in the evaluation light irradiation unit 20 is not limited to the configuration described above, and for example, the irradiation light source 21 and the objective lens 23 may be linearly arranged without using the reflection mirror 22. In the above configuration, an emission position of the light source element in the irradiation light source 21, a center position of the collimating lens, and a center position of the objective lens 23 are coaxially arranged on the same straight line. Further, in the dead cell number measurement unit 30, in the case in which the objective lens is used for the observation of the optical image of the object cell sample S, a center position of the observation objective lens is preferably arranged on the same straight line.

As described above, in the configuration in which the components of the irradiation optical system are linearly arranged, the number of components used in the evaluation light irradiation unit 20 is reduced, and thus, it is possible to reduce loss of the light and reduce a cost of the apparatus. In addition, in the above configuration, the optical path of the evaluation light is linearly formed, and thus, it is necessary to pay attention to the point that the height of the irradiation plane becomes high.

In addition, in the configuration of the evaluation light irradiation unit 20 illustrated in FIG. 1, the QCL element, the cooling mechanism, and the collimating lens constituting the irradiation light source 21 may be configured to be installed on an optical stage capable of integrally performing position adjustment. In this case, required accuracy in manufacturing the apparatus can be reduced. Further, similarly, an angle adjustment mechanism may be provided for the reflection mirror 22. Further, the objective lens 23 may be provided with an adjustment mechanism for the x axis direction, the y axis direction, and the z axis direction.

Further, in the case in which the lens is provided in the irradiation optical system, similarly to the collimating lens in the irradiation light source 21, it is preferable to select an appropriate material, and further, provide anti-reflection coating on both surfaces of the lens. Further, as the objective lens 23, a reflection type objective lens may be used. Further, as the objective lens 23, for example, a single lens such as an aspheric lens, a plano convex lens, or the like may be used instead of a configuration in which a plurality of lenses are used in combination. In addition, in order to achieve both a narrow irradiation spot of the evaluation light and a certain depth of focus, it is preferable to use the objective lens in which the plurality of lenses are used in combination.

The irradiation condition of the evaluation light on the object cell sample S in the evaluation light irradiation unit 20 will be described. The irradiation condition of the evaluation light for the irradiation region of the object cell sample S needs to be set such that the object correlation between the irradiation amount of the evaluation light and the dead cell number used for the evaluation of the object cell sample S can be acquired. Specifically, as described above, in the evaluation light irradiation unit 20, the configuration in which the N irradiation regions set by dividing the culture region in the object cell sample S are irradiated with the evaluation light with the irradiation amounts different from each other is used.

Further, in the evaluation light irradiation unit 20, it is preferable that the irradiation amount of the evaluation light is set by the irradiation condition including at least one of the irradiation time, the irradiation power, and the focusing condition of the evaluation light for the irradiation region. In this case, the irradiation condition capable of acquiring the object correlation between the irradiation amount of the evaluation light and the dead cell number used for the evaluation of the object cell sample S can be suitably realized by controlling the irradiation amount.

In the above configuration, in consideration of the fact that adjustment of the irradiation optical system is necessary to control and change the focusing condition, it is preferable to set the irradiation amount of the evaluation light by controlling one or both of the irradiation time and the irradiation power on the irradiation region of the evaluation light with the focusing condition being constant. In addition, in the case in which the focusing condition is changed, the irradiation power density of the evaluation light on the object cell sample S is changed, and the generation condition of the dead cells is changed. Hereinafter, as a specific example, setting of the irradiation amount in the case in which the burst pulse including the plurality of light pulses is used as the evaluation light will be described.

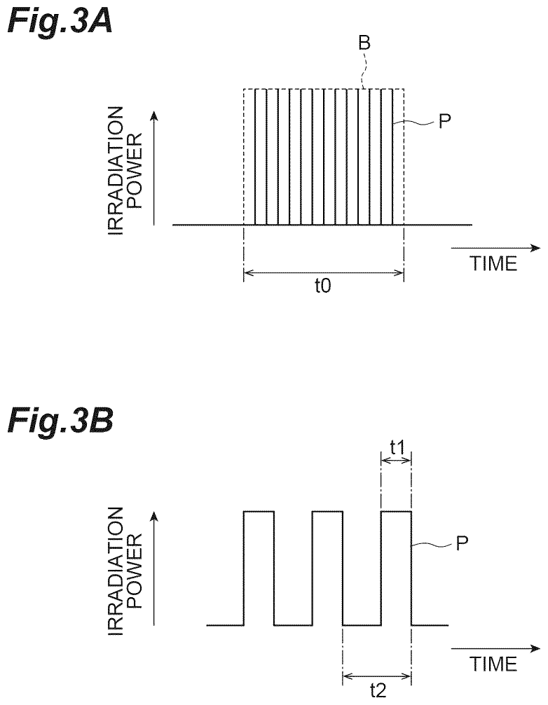

FIG. 3A and FIG. 3B are diagrams illustrating an example of the setting of the irradiation amount of the evaluation light supplied from the irradiation light source 21. FIG. 3A is a diagram illustrating the burst pulse supplied as the evaluation light from the irradiation light source 21. FIG. 3B is a diagram illustrating the light pulse included in the burst pulse by enlarging a part of the burst pulse illustrated in FIG. 3A.

As illustrated in FIG. 3A, the burst pulse includes the plurality of light pulses P, and is configured as the pulse having a range which is defined by an envelope B of a predetermined shape. In the burst pulse illustrated in FIG. 3A, the envelope B has a rectangular shape, and a time width t0 of the envelope B is set as the irradiation time in the case in which the above burst pulse is used as the evaluation light.

Further, in the plurality of light pulses P included in the burst pulse, as illustrated in FIG. 3B, a pulse width is set to t1, and a repetition period of the light pulses is set to t2. As the configuration of the burst pulse described above, specifically, for example, a configuration in which the pulse width is set to t1=100 μs and the repetition period is set to t2=1 ms can be used. In this case, a repetition frequency of the light pulses P in the burst pulse is set to 1 kHz.

In the burst pulse described above, as a method for changing the average irradiation power to be the irradiation power of the evaluation light, there are the following three methods. In the first method, an injection current applied to the light source element in the irradiation light source 21 and the repetition period t2 are fixed, and the pulse width t1 is changed. In the second method, the injection current applied to the light source element and the pulse width t1 are fixed, and the repetition period t2 is changed. In the third method, the pulse width t1 and the repetition period t2 are fixed, and the injection current applied to the light source element is changed. In the case of the third method, a pulse height value of the light pulse P constituting the burst pulse is changed by changing the injection current applied to the light source element.

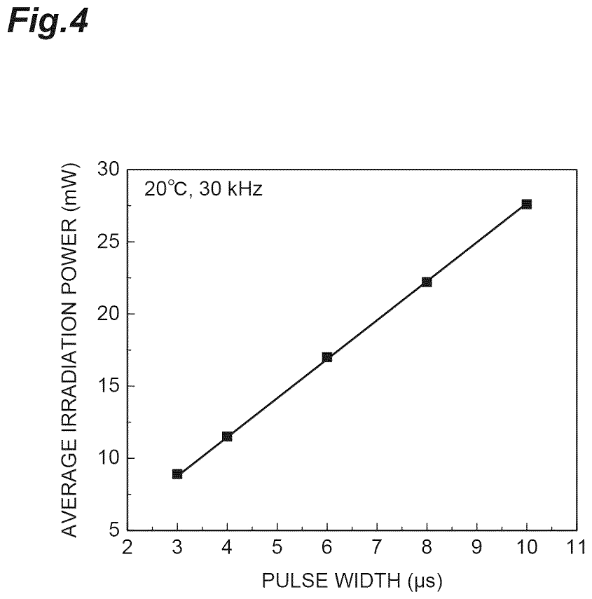

FIG. 4 is a graph showing an example of the setting of the irradiation power of the evaluation light supplied from the irradiation light source 21. In the graph of FIG. 4, the horizontal axis indicates the pulse width t1 (μs) of the light pulse P, and the vertical axis indicates the average irradiation power (mW).

In this case, the change of the average irradiation power when the pulse width t1 of the light pulse P is changed is shown under the conditions in which the temperature of the QCL in the irradiation light source 21 is set to 20° C., the injection current applied to the QCL is fixed, and the repetition frequency of the light pulses P in the burst pulse is fixed at 30 kHz. As described above, in the configuration in which the burst pulse is used as the evaluation light, the average irradiation power of the evaluation light can be controlled by changing any one of the injection current applied to the light source element, the pulse width of the light pulse P, and the repetition period (repetition frequency).

The irradiation condition of the evaluation light on the object cell sample S in the evaluation light irradiation unit 20 will be further described. In the irradiation of the evaluation light onto the object cell sample S, in order to suitably acquire the object correlation between the irradiation amount of the evaluation light and the dead cell number for the object cell sample S, the N irradiation regions (plurality of irradiation regions) are set by dividing the culture region in the object cell sample S, and further, the N irradiation regions are irradiated with the evaluation light supplied from the irradiation light source 21 with the irradiation amounts different from each other for the respective irradiation regions.

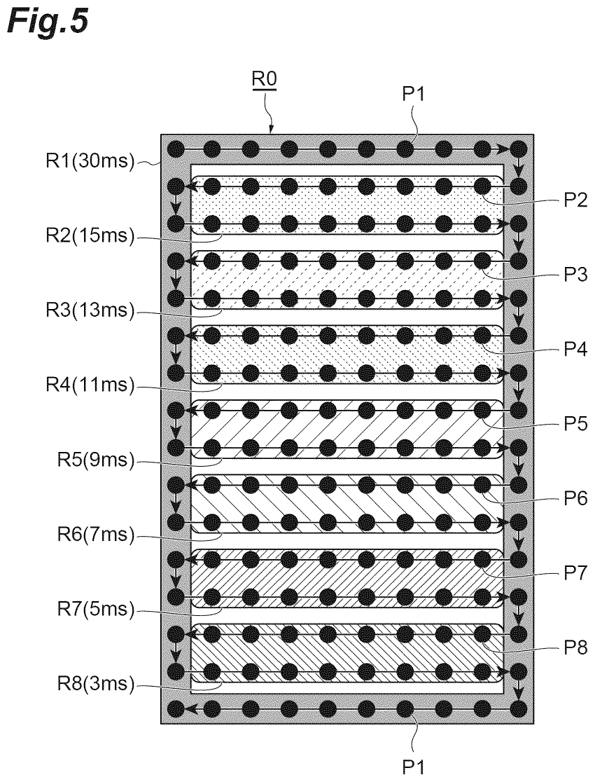

FIG. 5 is a diagram illustrating an example of the N irradiation regions set by dividing the culture region in the object cell sample S. In this case, the example in which it is set to N=8 and the eight irradiation regions R1 to R8 are set in the culture region R0 of the object cell sample S is illustrated. In the above irradiation regions R1 to R8, the irradiation region R1 is set as an outer peripheral region in the culture region R0. Further, the irradiation regions R2 to R8 are respectively set by dividing an inner portion of the irradiation region R1 such that the regions have substantially the same areas.

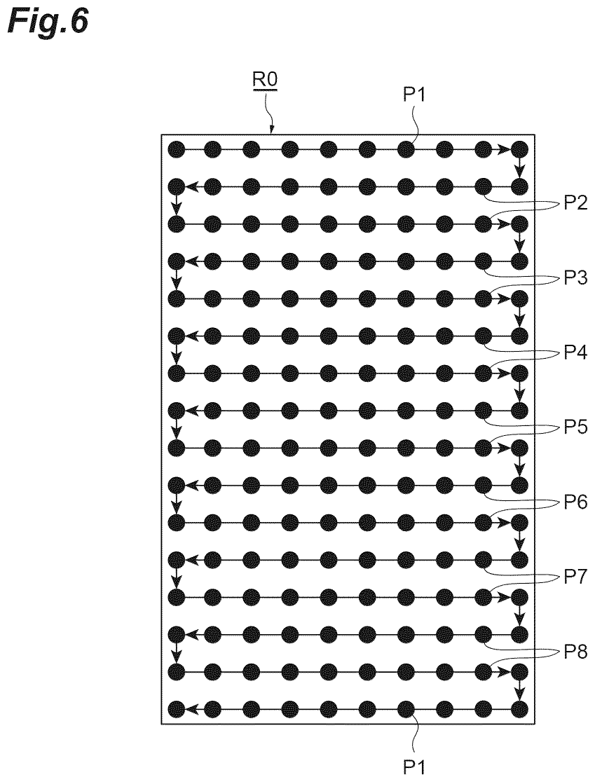

Further, in the configuration example illustrated in FIG. 5, the plurality of irradiation points for the irradiation with the evaluation light are set in each of the eight irradiation regions R1 to R8. FIG. 6 is a diagram illustrating the irradiation of the evaluation light for the irradiation points set in the eight irradiation regions R1 to R8 illustrated in FIG. 5. As illustrated in FIG. 5 and FIG. 6, in the present configuration example, the 48 irradiation points P1 are set for the irradiation region R1 of the outer peripheral region. Further, the 16 irradiation points P2 to P8 are set for each of the inner irradiation regions R2 to R8. The total number of irradiation points in the culture region R0 is 160.

Further, in the present configuration example, all the 160 irradiation points of the irradiation points P1 to P8 set for the eight irradiation regions R1 to R8 are arranged in the grid pattern (lattice pattern, two-dimensional array pattern) in the culture region R0, and as illustrated by an arrowed line in the configuration of FIG. 6, the irradiation points are sequentially subjected to focused irradiation with the evaluation light. According to the above configuration, the irradiation of the evaluation light on the object cell sample S for acquiring the object correlation can be suitably and efficiently performed. Further, as to the irradiation points P1 in the outer peripheral region, in order to clarify the grid of the irradiation region, it is preferable that the irradiation intensity or the irradiation time is set to an intensity or a time sufficient to reliably cause the cell death.

Further, in the configuration in which the plurality of irradiation points set in the object cell sample S are arranged in the grid pattern and irradiated with the evaluation light sequentially, it is possible to easily perform the change of the irradiation position of the evaluation light by moving the sample stage 18 in the xy plane, and the scanning of the respective irradiation points by the evaluation light according thereto. In the configuration example illustrated in FIG. 6, specifically, for 16 scanning lines each including the ten irradiation points and arranged in the vertical direction in the diagram, the irradiation points are scanned from left to right in each of the odd-numbered scanning lines from the top, and the irradiation points are scanned from right to left in each of the even-numbered scanning lines.

According to the above configuration, it is possible to reduce the movement distances of the sample stage 18 in the scanning of the plurality of irradiation points by the evaluation light. In general, it is preferable that, for the plurality of scanning lines arranged in the predetermined direction set for scanning the plurality of irradiation points arranged in the grid pattern, the scanning of the irradiation points from left to right and the scanning of the irradiation points from right to left are alternately performed in the odd-numbered scanning lines and the even-numbered scanning lines so that the scanning directions are set to be alternately different.

In the case in which the plurality of irradiation points are set in each of the irradiation regions in the object cell sample S as described above, it is preferable that the dead cell number measurement unit 30 measures the dead cell number at each of the plurality of irradiation points in each of the N irradiation regions. Further, it is preferable that the object correlation generation unit 52 of the culture state analysis unit 50 obtains the object correlation between the irradiation amount of the evaluation light in each irradiation region and an average value of the dead cell numbers at the plurality of irradiation points in each irradiation region for the N irradiation regions. As described above, by using the average value of the dead cell numbers at the plurality of irradiation points, it is possible to appropriately and accurately acquire the object correlation between the irradiation amount of the evaluation light and the dead cell number for the N irradiation regions with the plurality of irradiation points set in each of the irradiation regions.

In addition, as to the setting and change of the irradiation point and the irradiation position of the evaluation light in the evaluation light irradiation unit 20, in the configuration illustrated in FIG. 1, as described above, the irradiation position is set by driving the sample stage 18 in the xy plane by the stage drive unit 19, and further, the configuration is not limited thereto, and various configurations may be specifically used.

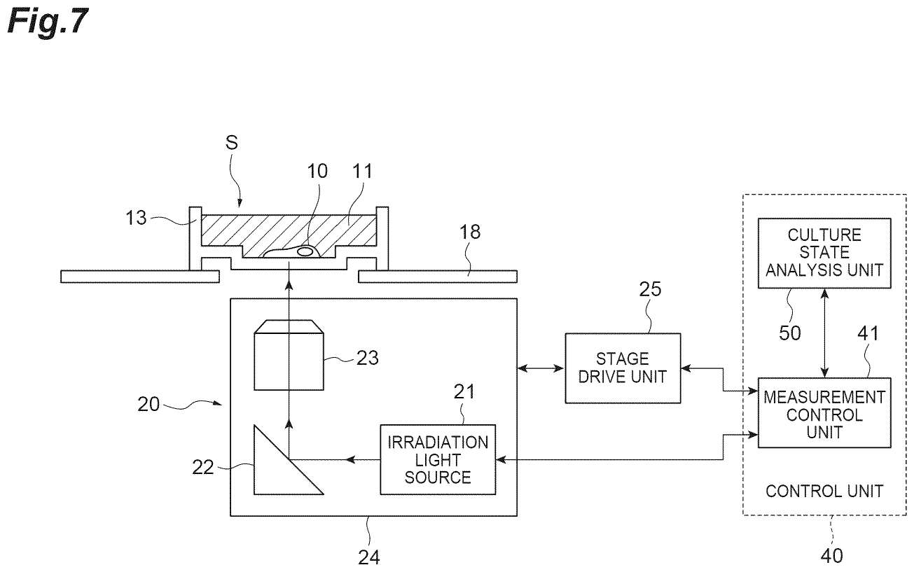

FIG. 7 is a diagram illustrating a configuration of the evaluation light irradiation unit 20 in the cell sample measurement apparatus 1A illustrated in FIG. 1 according to a first modification. In the configuration example illustrated in FIG. 7, the irradiation light source 21, the reflection mirror 22, and the objective lens 23 are installed on an optical system stage 24. Further, a stage drive unit 25 is provided for the optical system stage 24. The measurement control unit 41 can set and control the irradiation position of the evaluation light in the culture region of the object cell sample S by controlling the drive operation of the optical system stage 24 by the stage drive unit 25.

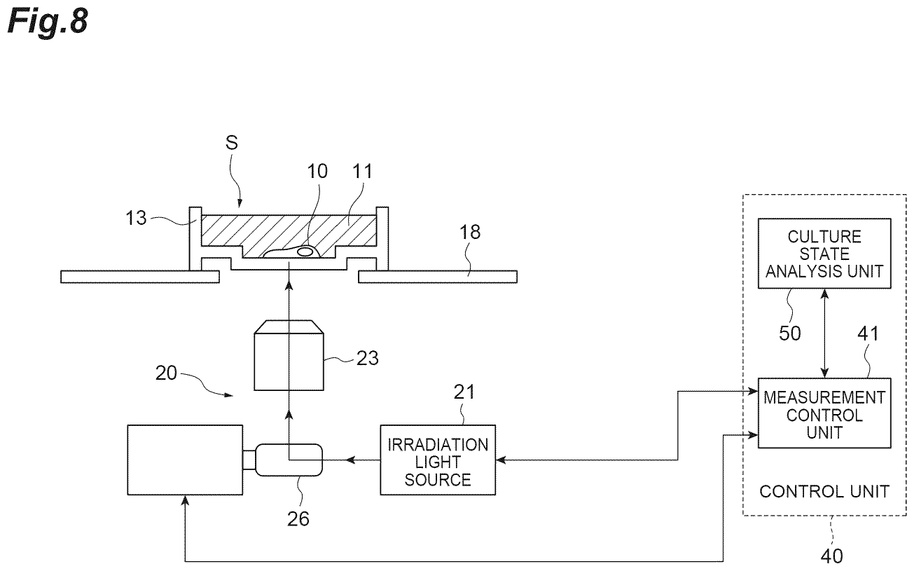

FIG. 8 is a diagram illustrating a configuration of the evaluation light irradiation unit 20 in the cell sample measurement apparatus 1A illustrated in FIG. 1 according to a second modification. In the configuration example illustrated in FIG. 8, a galvano mirror 26 is provided instead of the reflection mirror 22. The measurement control unit 41 can set and control the irradiation position of the evaluation light in the culture region of the object cell sample S by controlling the reflection direction of the evaluation light by the galvano mirror 26.

The measurement method of the dead cell number generated in the irradiation region of the object cell sample S in the dead cell number measurement unit 30 will be described. For the measurement of the dead cell number by the dead cell number measurement unit 30, a configuration can be used in which the dead cell number is measured by capturing an optical image of the object cell sample S stained with a staining agent for selectively staining the dead cells. According to the above configuration, it is possible to suitably measure the dead cell number generated according to the irradiation amount of the evaluation light for the irradiation region of the object cell sample S.

Further, as the staining agent for selectively staining the dead cells described above, for example, a fluorescent dye can be used. In this case, the dead cell number measurement unit 30 measures the dead cell number by capturing a fluorescent image generated by irradiating the object cell sample S stained with the fluorescent dye with excitation light having a predetermined wavelength. As the fluorescent dye described above, for example, a chemical material called propidium iodide, a fluorescent dye called CellEvent manufactured by Invitrogen, and the like can be used.

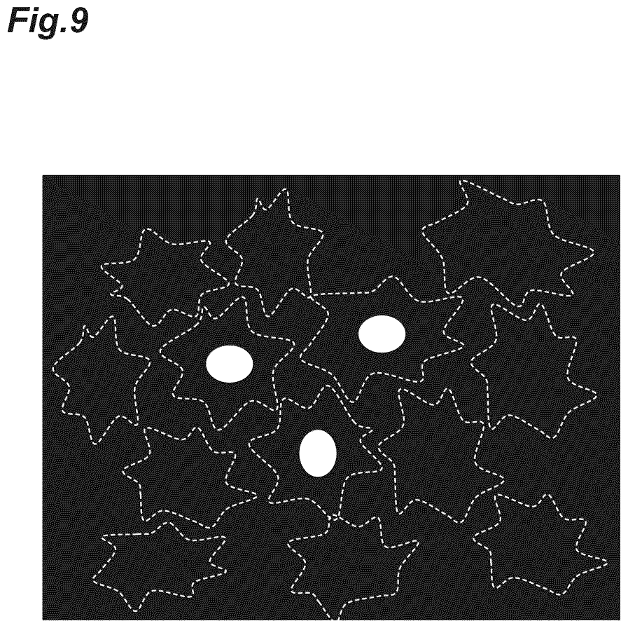

FIG. 9 is a diagram schematically illustrating the fluorescent image of the object cell sample S stained with the fluorescent dye for selectively staining the dead cells. Many fluorescent dyes for staining only the dead cells, in general, stain cell nuclei particularly strongly. Therefore, after acquiring the fluorescent image of the object cell sample S stained with the fluorescent dye, the dead cell number in the object cell sample S can be calculated by counting the number of stained cell nuclei by, for example, counting the dead cell number visually by an operator, or counting the dead cell number by using an automatic image analysis technique described in Non Patent Document 3 (N. Malpica et al., Cytometry Vol. 28 (1997), pp. 289-297).

In the counting of the dead cell number in the fluorescent image, for example, for the acquired fluorescent image, a binarized image in which a pixel with a fluorescence intensity exceeding a threshold value is set to 1, and a pixel with the fluorescence intensity equal to or less than the threshold value is set to 0 may be used. Further, as a method for more easily counting the dead cell number, it is also possible to use a method of referring to an area of a region of the dead cells. In this case, for example, the binarized image of the fluorescent image is generated as described above, and the number of pixels having the intensity of 1 is obtained in the region for counting the dead cell number. The above number of pixels corresponds to the area of the cell nuclei belonging to the dead cells.

Further, in addition to the calculation of the number of pixels described above, an average area of the cell nucleus is obtained. In the calculation of the area of the cell nucleus in this case, for example, a method can be used in which an operator manually measures the number of cell nuclei and the area for an image including a plurality of dead cells, and obtains the area of the cell nucleus by taking the average thereof. An estimated value of the dead cell number can be easily calculated by dividing the area (number of pixels) of the cell nuclei belonging to the dead cells by the average area (number of pixels) of the cell nucleus described above. According to the above method, the estimated value of the dead cell number included in each of the images can be easily obtained in a short time by the simple analysis algorithm, for example, even in the case in which the number of images is 100 or more.

As the staining agent for selectively staining the dead cells used in the measurement of the dead cell number in the object cell sample S, in addition to the fluorescent dye described above, for example, a staining agent for staining the cells in an absorptive manner, such as trypan blue or the like, can be used. Further, as to the measurement of the dead cell number, in addition to the method using the staining agent, for example, a method can be used in which images of the object cell sample S are acquired in time series, and cells with motion are counted as live cells and cells without motion are counted as dead cells.

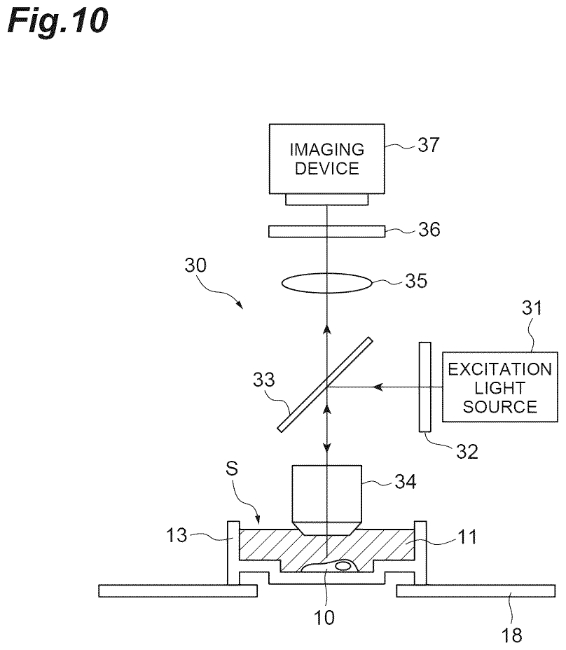

FIG. 10 is a diagram illustrating an example of a configuration of the dead cell number measurement unit 30 in the cell sample measurement apparatus 1A illustrated in FIG. 1. The configuration example illustrated in FIG. 10 illustrates a configuration of the dead cell number measurement unit 30 in the case of acquiring the fluorescent image of the object cell sample S stained with the fluorescent dye for selectively staining the dead cells. In the present configuration example, the dead cell number measurement unit 30 includes an excitation light source 31, an excitation filter 32, a dichroic mirror 33, an objective lens 34, an imaging lens 35, a fluorescence filter 36, and an imaging device 37 (see, for example, Patent Document 2).

The excitation light source 31 supplies the excitation light having the wavelength corresponding to the fluorescent dye used for staining of the object cell sample S. The excitation filter 32 selectively transmits, as the excitation light, light in a wavelength region including the wavelength used for the excitation of the fluorescent dye in the light supplied from the excitation light source 31, and for example, a bandpass filter may be used. The dichroic mirror 33 reflects the excitation light supplied from the excitation light source 31 through the excitation filter 32 to the objective lens 34.

The plurality of cells 10 stained with the fluorescent dye in the object cell sample S are irradiated with the excitation light reflected by the dichroic mirror 33 through the objective lens 34. As the objective lens 34, for example, a water immersion objective lens may be used.

The fluorescence generated by the fluorescent dye contained in the dead cells in the object cell sample S due to the irradiation of the excitation light passes through the objective lens 34, the dichroic mirror 33, the imaging lens 35, and the fluorescence filter 36 to reach the imaging device 37, and the fluorescent image of the object cell sample S is captured by the imaging device 37. The fluorescence filter 36 is a filter (excitation light cut filter) for cutting light in the wavelength region of the excitation light and selectively transmitting light in a wavelength region of the fluorescence in the light output from the object cell sample S, and for example, a bandpass filter or a notch filter may be used.

The operation, effect, and the like of the cell sample measurement apparatus 1A and the cell sample measurement method of the configuration described above will be described together with a specific example and measurement data. In the example, as the cells 10 in the object cell sample S, the C6 cells which are the rat glioma cell line (obtained from JCRB cell bank) were used. As the culture medium, the Dulbecco's modified Eagle's medium (DMEM culture medium) containing 10% fetal bovine serum (FBS), 4 mM L-glutamine, 100 U/mL penicillin, and 400 μg/mL streptomycin was used, and flask static culture was performed in an incubator (37° C., 5% CO₂). The DMEM culture medium was replaced every three days.

The C6 cells reaching 100% confluence were detached with 0.25% trypsin EDTA, resuspended in the DMEM culture medium, and seeded in the lumox dish (manufactured by Sarstedt) which is the culture dish. Static culture was carried out in the incubator for three days, and the cells reaching 100% confluence again were subjected to the irradiation experiment of the evaluation light. Further, three types of cell samples having the passage numbers different from each other were prepared as the cell sample S. The passage numbers in the three types of the cell samples are 45, 53, and 55, respectively.

As to the irradiation condition and the irradiation method of the evaluation light for the cell sample S in the evaluation light irradiation unit 20, as illustrated in FIG. 5 and FIG. 6, the configuration was used in which the eight irradiation regions R1 to R8 were set by dividing the culture region R0 of the cell sample S, and the plurality of irradiation points were set in each of the irradiation regions to perform the irradiation with the evaluation light. Further, an arrangement pitch of the plurality of irradiation points arranged in the grid pattern in the culture region R0 was set to 500 μm in both the vertical direction and the horizontal direction.

As to the irradiation of the evaluation light on the cell sample S, the DFB-QCL described above having the wavelength of 4.6 μm was used as the irradiation light source 21, and the focusing condition of the evaluation light was set to be fixed. Further, the burst pulse illustrated in FIG. 3A and FIG. 3B was used as the evaluation light, the repetition frequency of the light pulses in the burst pulse was fixed to 30 kHz, and the pulse width of the light pulse was fixed to 6 ps. The average irradiation power in this case was measured to be 16.5 mW (see FIG. 4).

Further, as to the irradiation time of the evaluation light, as illustrated in FIG. 5, the irradiation times for the irradiation regions were set to be times (irradiation amounts) different from each other, specifically, the irradiation time was set to be 30 ms in the irradiation region R1, 15 ms in the irradiation region R2, 13 ms in the irradiation region R3, 11 ms in the irradiation region R4, 9 ms in the irradiation region R5, 7 ms in the irradiation region R6, 5 ms in the irradiation region R7, and 3 ms in the irradiation region R8. Further, as to the measurement method of the dead cell number in the dead cell number measurement unit 30, the configuration was used in which the fluorescent image of the cell sample S stained with the fluorescent dye for selectively staining the dead cells was acquired as described above.

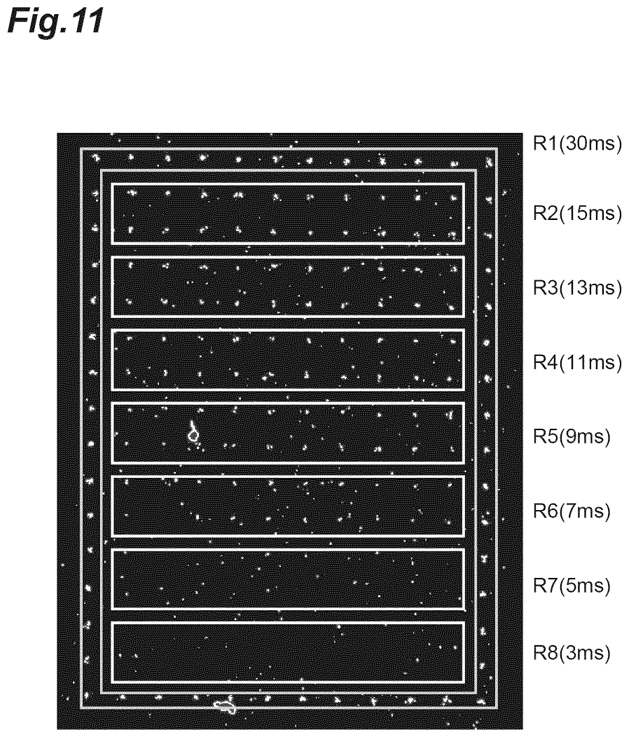

FIG. 11 is a diagram showing an example of the fluorescent image which is generated by irradiating the cell sample S stained with the fluorescent dye with the excitation light. The fluorescent image was observed after being left to stand in the incubator for four hours after the irradiation with the evaluation light. As can be seen from the fluorescent image shown in FIG. 11, the dead cells are generated at each of the plurality of irradiation points in each of the irradiation regions. Further, the numbers of dead cells generated at the respective irradiation points are different according to the irradiation amounts (irradiation times) of the evaluation light which are set to be different from each other for the respective irradiation regions, and the dead cell number increases as the irradiation time increases. In addition, the response of the cells to the light irradiation is not limited to an acute response immediately after the light irradiation, and may appear several hours after the irradiation, and therefore, the cells after the light irradiation are preferably placed in the incubator or the like, which is an environment suitable for the cells. The above environment is, for example, an environment with 37° C., humidity of 95%, and CO₂ concentration of 5%.

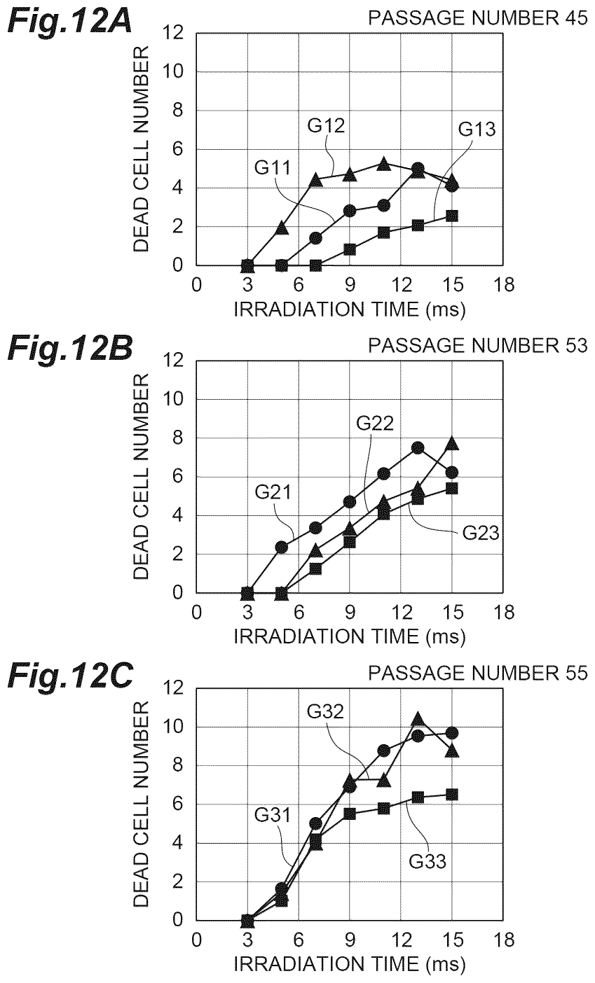

FIG. 12A to FIG. 12C are graphs each showing the correlation between the irradiation time of the evaluation light and the dead cell number for each of the cell samples S with the passage numbers 45, 53, and 55. In each of the graphs of FIG. 12A to FIG. 12C, the horizontal axis indicates the irradiation time (ms) of the evaluation light, and the vertical axis indicates the dead cell number (the number of dead cells). The dead cell number at each irradiation time indicates the average value of the dead cell numbers which are measured at the plurality of irradiation points set in the corresponding irradiation region.

Further, three cell samples were prepared for each of the three types of the cell samples with the passage numbers of 45, 53, and 55, and the measurements were performed. The graph of FIG. 12A shows the correlation for the cell samples with the passage number 45, and the measurement results for the three cell samples are respectively shown by graphs G11 to G13. The graph of FIG. 12B shows the correlation for the cell samples with the passage number 53, and the measurement results for the three cell samples are respectively shown by graphs G21 to G23. The graph of FIG. 12C shows the correlation for the cell samples with the passage number 55, and the measurement results for the three cell samples are respectively shown by graphs G31 to G33.

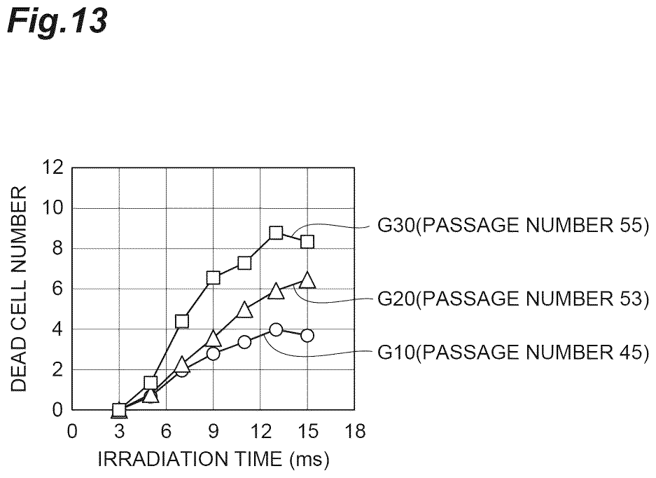

FIG. 13 is a graph showing the correlation between the irradiation time of the evaluation light and the dead cell number for each of the cell samples S with the passage numbers 45, 53, and 55. In FIG. 13, a graph G10 represents an average value of the graphs G11 to G13 obtained for the cell samples with the passage number 45 shown in FIG. 12A. A graph G20 represents an average value of the graphs G21 to G23 obtained for the cell samples with the passage number 53 shown in FIG. 12B. A graph G30 represents an average value of the graphs G31 to G33 obtained for the cell samples with the passage number 55 shown in FIG. 12C.

As shown in the respective graphs of FIG. 12A to FIG. 12C and FIG. 13, the correlations between the irradiation time (irradiation amount) of the evaluation light and the dead cell number acquired respectively for the cell samples are different depending on the passage number which is the culture state of the cell sample. Therefore, the culture state, such as the passage number or the like, of the cell sample can be measured and evaluated by using the correlation described above as the index.

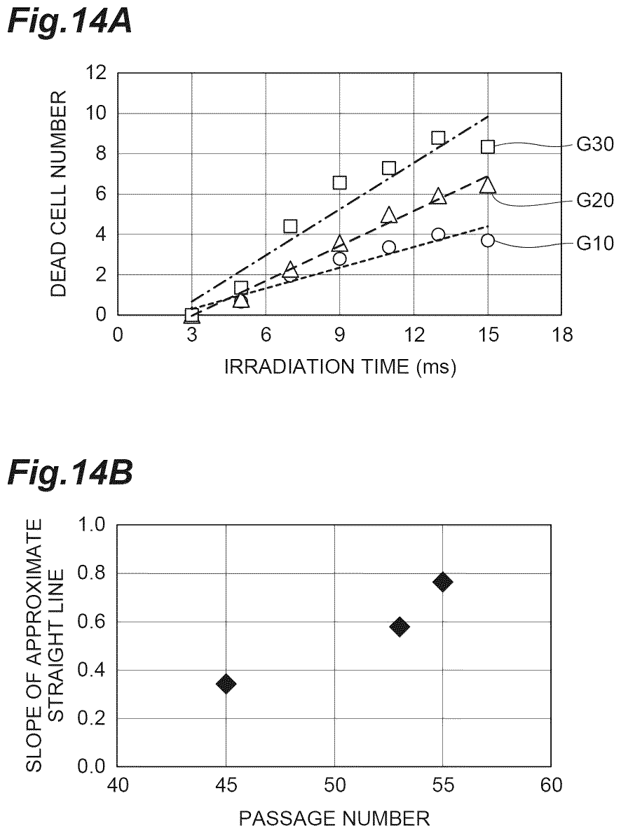

FIG. 14A and FIG. 14B are graphs showing a relationship between a slope of an approximate straight line, which is a parameter representing the approximate straight line of the correlation between the irradiation time of the evaluation light and the dead cell number, and the passage number, which is the culture state of the cell sample S. FIG. 14A is a graph showing the correlation between the irradiation time of the evaluation light and the dead cell number for each of the cell samples of the passage numbers 45, 53, and 55, and shows the graphs G10 to G30 same as the graphs of FIG. 13, and the approximate straight lines of the graphs. Further, FIG. 14B is a graph showing the relationship between the slope of the approximate straight line shown in FIG. 14A and the passage number.

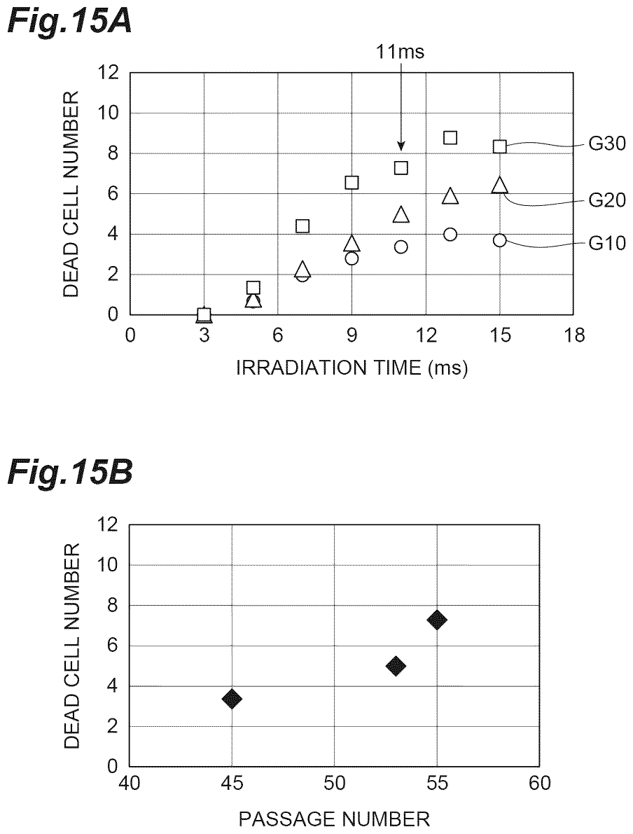

FIG. 15A and FIG. 15B are graphs showing a relationship between a dead cell number at a predetermined irradiation time (predetermined irradiation amount) in the correlation between the irradiation time of the evaluation light and the dead cell number, and the passage number which is the culture state of the cell sample S. FIG. 15A is a graph showing the correlation between the irradiation time of the evaluation light and the dead cell number for each of the cell samples of the passage numbers 45, 53, and 55, and shows the graphs G10 to G30 same as the graphs of FIG. 13, and the time of 11 ms set as the predetermined irradiation time for the correlation. Further, FIG. 15B is a graph showing the relationship between the dead cell number at the irradiation time of 11 ms shown in FIG. 15A and the passage number.

As shown in the respective graphs of FIG. 14A and FIG. 14B, and FIG. 15A and FIG. 15B, in the correlation between the irradiation amount of the evaluation light and the dead cell number for each cell sample, the parameter representing the approximate straight line or the approximate curve of the correlation (for example, the slope of the approximate straight line), or the dead cell number at the predetermined irradiation amount (for example, the predetermined irradiation time) can be used as the index of the evaluation of the culture state. Further, depending on the specific data situation of the correlation, a threshold irradiation amount for generating the dead cells may be used as the index of the evaluation.

In the above example, the correlation in the case in which the irradiation power and the focusing condition of the evaluation light are set to be fixed and the irradiation time is changed is shown, and in addition, for example, in the case in which the irradiation time and the focusing condition are set to be fixed and the irradiation power is changed, or in the case in which the focusing condition is set to be fixed and both the irradiation time and the irradiation power are changed, the correlation between the irradiation amount of the evaluation light and the dead cell number serving as the index of the evaluation of the culture state of the cell sample can be similarly acquired.

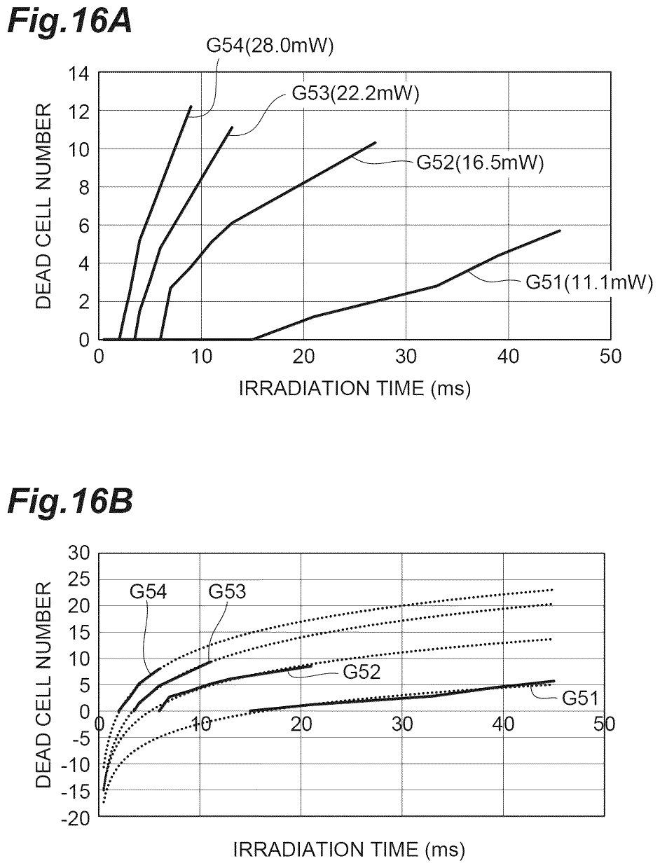

FIG. 16A and FIG. 16B are graphs each showing the correlation between the irradiation time and the irradiation power of the evaluation light and the dead cell number. In each of the graphs of FIG. 16A and FIG. 16B, the horizontal axis indicates the irradiation time (ms) of the evaluation light, and the vertical axis indicates the dead cell number. Further, in each of FIG. 16A and FIG. 16B, a graph G51 represents the correlation at the average irradiation power of 11.1 mW, a graph G52 represents the correlation at the average irradiation power of 16.5 mW, a graph G53 represents the correlation at the average irradiation power of 22.2 mW, and a graph G54 represents the correlation at the average irradiation power of 28.0 mW.

As shown in the respective graphs of FIG. 16A and FIG. 16B, in the case in which the focusing condition of the evaluation light is set to be fixed, the dead cell number generated in the irradiation region changes according to both the irradiation time and the irradiation power of the evaluation light. For example, when the average irradiation power of the evaluation light is set to P, and the irradiation time of the evaluation light is set to τ, the dead cell number can be represented by f(P, τ) as a function of these. In addition, in FIG. 16B, the approximate curve using the logarithm of each graph is also shown.

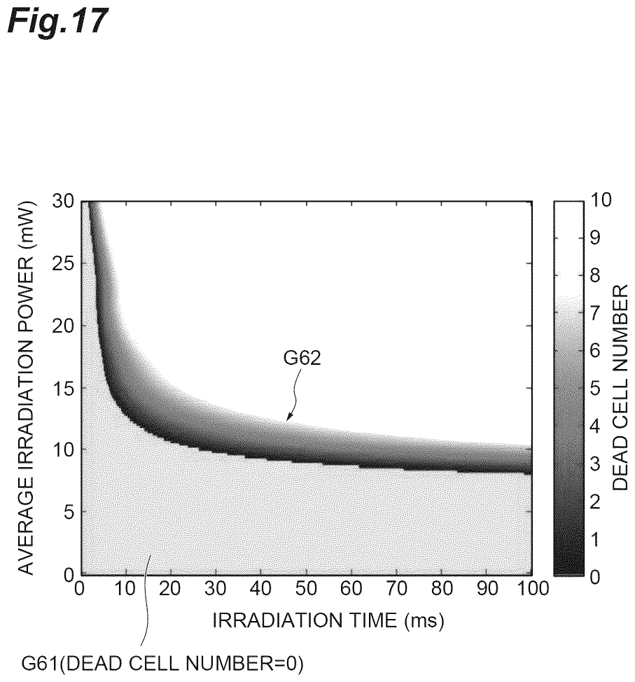

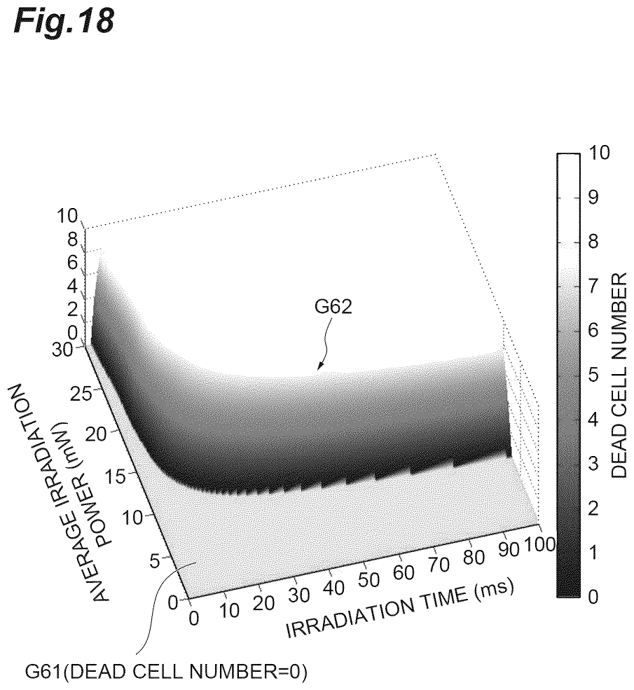

FIG. 17 and FIG. 18 are graphs each showing the correlation between the irradiation time and the average irradiation power of the evaluation light and the dead cell number. In the graph of FIG. 17, the horizontal axis indicates the irradiation time (ms) of the evaluation light, and the vertical axis indicates the average irradiation power (mW) of the evaluation light. In FIG. 17, a region G61 represents a region in which the dead cell number=0. Further, a region G62 represents a region in which the dead cells are generated, and the dead cell number is shown by grayscale. Further, the graph of FIG. 18 is a three-dimensional representation of the graph of FIG. 17.

In the graph of the dead cell number f(P, τ) in which the irradiation time τ and the irradiation power P are variables as described above, in the case in which a cross section is taken with the irradiation power P being fixed, a graph representing the correlation between the irradiation time τ of the evaluation light and the dead cell number is obtained. Further, in the case in which a cross section is taken with the irradiation time τ being fixed, a graph representing the correlation between the irradiation power P of the evaluation light and the dead cell number is obtained. As described above, in the evaluation of the culture state of the cell sample S, it is also possible to use the correlation between both the irradiation time and the irradiation power of the evaluation light and the dead cell number.

In addition, when the evaluation of the culture state of the object cell sample S is performed, for the comparison between the reference correlation and the object correlation, various methods may be specifically used. For example, in the case in which the single reference correlation is prepared, the evaluation can be performed by using a magnitude relationship between the reference correlation and the object correlation. Further, in the case in which the plurality of reference correlations are prepared, the evaluation can be performed by referring to the reference correlation closest to the object correlation out of the plurality of reference correlations. Further, for example, for the plurality of reference correlations, a relational formula or a lookup table representing a relationship between a parameter serving as the index such as the slope of the approximate straight line of the correlation or an intercept on the horizontal axis or the vertical axis of the approximate straight line, and the passage number may be prepared, and the evaluation may be performed by using the relational formula or the lookup table described above.

The cell sample measurement method and the cell sample measurement apparatus are not limited to the embodiments and configuration examples described above, and may be modified in various ways. For example, the configuration of each of the evaluation light irradiation unit 20 and the dead cell number measurement unit 30 is not limited to the configuration described above, and various configurations may be specifically used.

The cell sample measurement method of a first aspect according to the above embodiment includes (1) a preparation step of placing an object cell sample of a measurement object containing a plurality of cells cultured on a culture dish on a sample stage; (2) an evaluation light irradiation step of irradiating N irradiation regions (N is an integer of 2 or more) set by dividing a culture region of the plurality of cells in the object cell sample with evaluation light having a wavelength in a mid-infrared region supplied from an irradiation light source with irradiation amounts different from each other; (3) a dead cell number measurement step of measuring a dead cell number generated according to the irradiation amount of the evaluation light in each of the N irradiation regions of the object cell sample; and (4) a culture state analysis step of obtaining, for the object cell sample, an object correlation between the irradiation amount of the evaluation light and the dead cell number for the N irradiation regions serving as an index of evaluation of a culture state of the object cell sample.

The cell sample measurement apparatus of a first aspect according to the above embodiment includes (1) a sample stage for placing an object cell sample of a measurement object containing a plurality of cells cultured on a culture dish; (2) an evaluation light irradiation unit for irradiating N irradiation regions (N is an integer of 2 or more) set by dividing a culture region of the plurality of cells in the object cell sample with evaluation light having a wavelength in a mid-infrared region supplied from an irradiation light source with irradiation amounts different from each other; (3) a dead cell number measurement unit for measuring a dead cell number generated according to the irradiation amount of the evaluation light in each of the N irradiation regions of the object cell sample; and (4) a culture state analysis unit for obtaining, for the object cell sample, an object correlation between the irradiation amount of the evaluation light and the dead cell number for the N irradiation regions serving as an index of evaluation of a culture state of the object cell sample.

In the cell sample measurement method of a second aspect, in the configuration of the first aspect, in the evaluation light irradiation step, a plurality of irradiation points may be set for irradiating with the evaluation light in each of the N irradiation regions, and in the dead cell number measurement step, the dead cell number may be measured at each of the plurality of irradiation points in each of the N irradiation regions.

In the cell sample measurement apparatus of a second aspect, in the configuration of the first aspect, in the evaluation light irradiation unit, a plurality of irradiation points may be set for irradiating with the evaluation light in each of the N irradiation regions, and the dead cell number measurement unit may measure the dead cell number at each of the plurality of irradiation points in each of the N irradiation regions.

As described above, the plurality of irradiation points are set in each of the N irradiation regions set in the culture region, and the irradiation of the evaluation light and the measurement of the dead cell number are performed at each of the irradiation points, and thus, the object correlation between the irradiation amount of the evaluation light and the dead cell number can be suitably and accurately measured.

In the cell sample measurement method of a third aspect, in the configuration of the second aspect, in the evaluation light irradiation step, all the irradiation points set in the N irradiation regions may be arranged in a grid pattern in the culture region, and the irradiation points may be sequentially irradiated with the evaluation light.

In the cell sample measurement apparatus of a third aspect, in the configuration of the second aspect, in the evaluation light irradiation unit, all the irradiation points set in the N irradiation regions may be arranged in a grid pattern in the culture region, and the irradiation points may be sequentially irradiated with the evaluation light.

As described above, the irradiation points set in the culture region are arranged in the grid pattern, and the irradiation of the evaluation light is performed, and thus, the irradiation of the evaluation light on the object cell sample for acquiring the object correlation can be suitably and efficiently performed. Further, according to the above configuration, it is possible to easily perform the change of the irradiation position of the evaluation light, and the scanning of the respective irradiation points by the evaluation light according thereto.

In the cell sample measurement method of a fourth aspect, in the configuration of the second or third aspect, in the culture state analysis step, the object correlation between the irradiation amount of the evaluation light in each irradiation region and an average value of the dead cell numbers at the plurality of irradiation points in each irradiation region for the N irradiation regions may be obtained.

In the cell sample measurement apparatus of a fourth aspect, in the configuration of the second or third aspect, the culture state analysis unit may obtain the object correlation between the irradiation amount of the evaluation light in each irradiation region and an average value of the dead cell numbers at the plurality of irradiation points in each irradiation region for the N irradiation regions.

As described above, the average value of the dead cell numbers at the plurality of irradiation points is used, and thus, it is possible to suitably and accurately acquire the object correlation between the irradiation amount of the evaluation light and the dead cell number for the N irradiation regions with the plurality of irradiation points set in each irradiation region.

In the cell sample measurement method of a fifth aspect, in the configuration of any one of the first to fourth aspects, in the dead cell number measurement step, the dead cell number may be measured by capturing an optical image of the object cell sample stained with a staining agent for selectively staining dead cells.

In the cell sample measurement apparatus of a fifth aspect, in the configuration of any one of the first to fourth aspects, the dead cell number measurement unit may measure the dead cell number by capturing an optical image of the object cell sample stained with a staining agent for selectively staining dead cells.

In the cell sample measurement method of a sixth aspect, in the configuration of the fifth aspect, the staining agent may be a fluorescent dye, and in the dead cell number measurement step, the dead cell number may be measured by capturing a fluorescent image generated by irradiating the object cell sample stained with the fluorescent dye with excitation light.

In the cell sample measurement apparatus of a sixth aspect, in the configuration of the fifth aspect, the staining agent may be a fluorescent dye, and the dead cell number measurement unit may measure the dead cell number by capturing a fluorescent image generated by irradiating the object cell sample stained with the fluorescent dye with excitation light.

As described above, by using the staining agent for selectively staining the dead cells, it is possible to suitably measure the dead cell number generated according to the irradiation amount of the evaluation light for the irradiation region of the object cell sample. Further, as the above staining agent, for example, as described above, the fluorescent dye can be preferably used.

In the cell sample measurement method of a seventh aspect, in the configuration of any one of the first to sixth aspects, in the evaluation light irradiation step, the irradiation amount of the evaluation light may be set by an irradiation condition including at least one of an irradiation time, an irradiation power, and a focusing condition of the evaluation light for the irradiation region.

In the cell sample measurement apparatus of a seventh aspect, in the configuration of any one of the first to sixth aspects, in the evaluation light irradiation unit, the irradiation amount of the evaluation light may be set by an irradiation condition including at least one of an irradiation time, an irradiation power, and a focusing condition of the evaluation light for the irradiation region.

As described above, for the irradiation of the evaluation light on the irradiation region of the object cell sample, by setting and controlling at least one of the irradiation time, the irradiation power, and the focusing condition of the evaluation light, the irradiation condition capable of acquiring the object correlation between the irradiation amount of the evaluation light and the dead cell number used for the evaluation of the object cell sample can be suitably realized.

In the cell sample measurement method of an eighth aspect, in the configuration of any one of the first to seventh aspects, in the evaluation light irradiation step, the wavelength of the evaluation light may be set in a wavelength region of 3 μm or more and 20 μm or less.

In the cell sample measurement apparatus of an eighth aspect, in the configuration of any one of the first to seventh aspects, in the evaluation light irradiation unit, the wavelength of the evaluation light may be set in a wavelength region of 3 μm or more and 20 μm or less.

As described above, by setting the wavelength of the evaluation light in the mid-infrared region used for the evaluation of the culture state of the object cell sample, for example, within the wavelength region described above, the object correlation between the irradiation amount of the evaluation light and the dead cell number serving as the index of the evaluation can be suitably acquired.

In the cell sample measurement method of a ninth aspect, in the configuration of any one of the first to eighth aspects, in the evaluation light irradiation step, the irradiation light source for supplying the evaluation light may include a quantum cascade laser.

In the cell sample measurement apparatus of a ninth aspect, in the configuration of any one of the first to eighth aspects, in the evaluation light irradiation unit, the irradiation light source for supplying the evaluation light may include a quantum cascade laser.

As described above, by using the quantum cascade laser as the irradiation light source for supplying the evaluation light, the irradiation of the evaluation light having the wavelength in the mid-infrared region on the object cell sample can be suitably performed.

In the cell sample measurement method of a tenth aspect, in the configuration of any one of the first to ninth aspects, in the evaluation light irradiation step, an irradiation position of the evaluation light in the culture region may be set by driving the sample stage on which the object cell sample is placed in a plane intersecting with an irradiation optical axis of the evaluation light.

In the cell sample measurement apparatus of a tenth aspect, in the configuration of any one of the first to ninth aspects, in the evaluation light irradiation unit, an irradiation position of the evaluation light in the culture region may be set by driving the sample stage on which the object cell sample is placed in a plane intersecting with an irradiation optical axis of the evaluation light.

As described above, by using the configuration in which the sample stage is moved in the plane intersecting with the irradiation optical axis of the evaluation light (for example, in the plane perpendicular to the irradiation optical axis), the irradiation of the evaluation light on each of the N irradiation regions set in the culture region can be suitably performed.

The present invention can be used as a cell sample measurement method and a cell sample measurement apparatus capable of suitably performing a measurement for a culture state of a cell sample containing a plurality of cells.

From the invention thus described, it will be obvious that the invention may be varied in many ways. Such variations are not to be regarded as a departure from the spirit and scope of the invention, and all such modifications as would be obvious to one skilled in the art are intended for inclusion within the scope of the following claims.

What is claimed is:

1. A cell sample measurement method comprising:
a preparation step of placing an object cell sample of a measurement object containing a plurality of cells cultured on a culture dish on a sample stage;
an evaluation light irradiation step of irradiating N irradiation regions (N is an integer of 2 or more) set by dividing a culture region of the plurality of cells in the object cell sample with evaluation light having a wavelength in a mid-infrared region supplied from an irradiation light source with irradiation amounts different from each other;
a dead cell number measurement step of measuring a dead cell number generated according to the irradiation amount of the evaluation light in each of the N irradiation regions of the object cell sample; and
a culture state analysis step of obtaining, for the object cell sample, an object correlation between the irradiation amount of the evaluation light and the dead cell number for the N irradiation regions serving as an index of evaluation of a culture state of the object cell sample.

2. The cell sample measurement method according to claim 1, wherein in the evaluation light irradiation step, a plurality of irradiation points are set for irradiating with the evaluation light in each of the N irradiation regions, and
in the dead cell number measurement step, the dead cell number is measured at each of the plurality of irradiation points in each of the N irradiation regions.

3. The cell sample measurement method according to claim 2, wherein in the evaluation light irradiation step, all the irradiation points set in the N irradiation regions are arranged in a grid pattern in the culture region, and the irradiation points are sequentially irradiated with the evaluation light.

4. The cell sample measurement method according to claim 2, wherein in the culture state analysis step, the object correlation between the irradiation amount of the evaluation light in each irradiation region and an average value of the dead cell numbers at the plurality of irradiation points in each irradiation region for the N irradiation regions is obtained.

5. The cell sample measurement method according to claim 1, wherein in the dead cell number measurement step, the dead cell number is measured by capturing an optical image of the object cell sample stained with a staining agent for selectively staining dead cells.

6. The cell sample measurement method according to claim 5, wherein the staining agent is a fluorescent dye, and
in the dead cell number measurement step, the dead cell number is measured by capturing a fluorescent image generated by irradiating the object cell sample stained with the fluorescent dye with excitation light.

7. The cell sample measurement method according to claim 1, wherein in the evaluation light irradiation step, the irradiation amount of the evaluation light is set by an irradiation condition including at least one of an irradiation time, an irradiation power, and a focusing condition of the evaluation light for the irradiation region.

8. The cell sample measurement method according to claim 1, wherein in the evaluation light irradiation step, the wavelength of the evaluation light is set in a wavelength region of 3 μm or more and 20 μm or less.

9. The cell sample measurement method according to claim 1, wherein in the evaluation light irradiation step, the irradiation light source for supplying the evaluation light includes a quantum cascade laser.

10. The cell sample measurement method according to claim 1, wherein in the evaluation light irradiation step, an irradiation position of the evaluation light in the culture region is set by driving the sample stage on which the object cell sample is placed in a plane intersecting with an irradiation optical axis of the evaluation light.

11. A cell sample measurement apparatus comprising:
a sample stage configured to place an object cell sample of a measurement object containing a plurality of cells cultured on a culture dish;
an evaluation light irradiation unit configured to irradiate N irradiation regions (N is an integer of 2 or more) set by dividing a culture region of the plurality of cells in the object cell sample with evaluation light having a wavelength in a mid-infrared region supplied from an irradiation light source with irradiation amounts different from each other;
a dead cell number measurement unit configured to measure a dead cell number generated according to the irradiation amount of the evaluation light in each of the N irradiation regions of the object cell sample; and
a culture state analysis unit configured to obtain, for the object cell sample, an object correlation between the irradiation amount of the evaluation light and the dead cell number for the N irradiation regions serving as an index of evaluation of a culture state of the object cell sample.

12. The cell sample measurement apparatus according to claim 11, wherein in the evaluation light irradiation unit, a plurality of irradiation points are set for irradiating with the evaluation light in each of the N irradiation regions, and
the dead cell number measurement unit is configured to measure the dead cell number at each of the plurality of irradiation points in each of the N irradiation regions.

13. The cell sample measurement apparatus according to claim 12, wherein in the evaluation light irradiation unit, all the irradiation points set in the N irradiation regions are arranged in a grid pattern in the culture region, and the irradiation points are sequentially irradiated with the evaluation light.

14. The cell sample measurement apparatus according to claim 12, wherein the culture state analysis unit is configured to obtain the object correlation between the irradiation amount of the evaluation light in each irradiation region and an average value of the dead cell numbers at the plurality of irradiation points in each irradiation region for the N irradiation regions.

15. The cell sample measurement apparatus according to claim 11, wherein the dead cell number measurement unit is configured to measure the dead cell number by capturing an optical image of the object cell sample stained with a staining agent for selectively staining dead cells.

16. The cell sample measurement apparatus according to claim 15, wherein the staining agent is a fluorescent dye, and
the dead cell number measurement unit is configured to measure the dead cell number by capturing a fluorescent image generated by irradiating the object cell sample stained with the fluorescent dye with excitation light.

17. The cell sample measurement apparatus according to claim 11, wherein in the evaluation light irradiation unit, the irradiation amount of the evaluation light is set by an irradiation condition including at least one of an irradiation time, an irradiation power, and a focusing condition of the evaluation light for the irradiation region.

18. The cell sample measurement apparatus according to claim 11, wherein in the evaluation light irradiation unit, the wavelength of the evaluation light is set in a wavelength region of 3 μm or more and 20 μm or less.

19. The cell sample measurement apparatus according to claim 11, wherein in the evaluation light irradiation unit, the irradiation light source for supplying the evaluation light includes a quantum cascade laser.

20. The cell sample measurement apparatus according to claim 11, wherein in the evaluation light irradiation unit, an irradiation position of the evaluation light in the culture region is set by driving the sample stage on which the object cell sample is placed in a plane intersecting with an irradiation optical axis of the evaluation light.
